(19) United States
(12) Patent Application Publication
(10) Pub. No.: US 2025/0221709 A1
(43) Pub. Date: Jul. 10, 2025

(54) TISSUE ANCHORS AND METHODS OF CONSTRUCTION THEREOF

(71) Applicant: Edwards Lifesciences Innovation (Israel) Ltd., Caesarea (IL)

(72) Inventors: Carmel Peleg, Pardes Hana-Karkur (IL); Haim Brauon, Beit Dagan (IL); Itay Avinathan, Tel-Aviv (IL); Ofry Efraim Yossef, Tel-Aviv (IL); Andrew Charles May, Irvine, CA (US)

(21) Appl. No.: 19/093,156

(22) Filed: Mar. 27, 2025

Related U.S. Application Data
(63) Continuation of application No. PCT/IB2023/059125, filed on Sep. 14, 2023.
(60) Provisional application No. 63/488,157, filed on Mar. 2, 2023.
(60) Provisional application No. 63/410,986, filed on Sep. 28, 2022.

(51) Int. Cl. A61B 17/08

(52) U.S. Cl. CPC A61B 17/08

(57) ABSTRACT

A kit for manufacture of a tissue anchor comprises (a) a helical tissue-engaging element, including a helical portion, and a transverse bar extending from an end of the helical portion, across a chord of a circular projection of the helical portion, and (b) a head, comprising first and second arms, each of the first and second arms including: (A) a root portion; and (B) a transverse portion extending from the root portion, each terminating at an arm end, such that the arm ends of the transverse portions of the first and second arms are directed away from each other, and the transverse portions are substantially parallel to one another; and (iv) a gap formed between the transverse portions of the first and second arms, substantially parallel to the transverse portions of the first and second arms. Other embodiments are also described.

### CROSS-REFERENCE TO RELATED APPLICATIONS

The present application is a continuation of International Patent Application No. PCT/IB2023/059125, filed Sep. 14, 2023, which claims the benefit of U.S. Patent Application No. 63/410,986, filed Sep. 28, 2022, and of U.S. Patent Application No. 63/488,157, filed Mar. 2, 2023, the entire disclosures all of which are incorporated by reference for all purposes.

### BACKGROUND

In recent years, the number of transcatheter cardiac surgeries and medical processes in mammalian, and specifically human, hearts, has been increasing. Many such procedures require insertion of a tissue anchor into the cardiac tissue. Tissue anchors can include an anchor head, which remains outside of the tissue, and a tissue-engaging element which is inserted into the tissue.

In many prior art tissue anchors, the anchor head is secured to the tissue-engaging element by welding. Welding may be unreliable under fatigue loading and may be less predictable in its behavior under strained conditions.

There is thus a need for improved techniques and devices for attaching the tissue-engaging element and the head of a tissue anchor to each other, without requiring welding, in a manner that is reliable, predictable, and safe.

### SUMMARY

This summary is meant to provide some examples and is not intended to be limiting of the scope of the disclosure in any way. For example, any feature included in an example of this summary is not required by the claims, unless the claims explicitly recite the features. Also, the features described can be combined in a variety of ways. Various features and steps as described elsewhere in this disclosure can be included in the examples summarized here.

Various implementations and examples herein relate to tissue anchors for implantation in tissue of a subject or simulation, and to methods of manufacture thereof, particularly methods of manufacture that reduce a requirement for, and/or dependence on, welding.

In some implementations, a tissue anchor includes a tissue-engaging element, and an anchor head. In some implementations, the tissue-engaging element can include a helical portion, and/or a transverse bar extending across the helical portion.

In some implementations, the transverse bar can be adapted to be received in a bore, extending transversely through the anchor head, thereby securing the tissue-engaging element to the anchor head.

In some implementations, a first coil of the helical portion can be wrapped around a neck portion of the anchor head.

In some implementations, the tissue anchor can further include a hollow cap, adapted to be secured to the anchor head, but pins extending through the hollow cap and through a circumferential groove in the anchor head. In some implementations, the cap obstructs motion of the transverse bar out of the bore, thereby further securing the tissue-engaging element to the anchor head.

In some implementations, an anchor head includes two arms, extending out of a face of the anchor head and including transverse portions parallel to the face, such that each arm defines a cavity. In some implementations, the two arms disposed in opposite directions to one another with their transverse portions parallel to one another and having a gap therebetween.

In some implementations, when manufacturing the tissue anchor, the transverse bar of the helical portion is inserted into the gap, and the tissue-engaging element is rotated such that the transverse bar becomes disposed between the transverse portions of the two arms, and the face of the anchor head. In some implementations, the transverse portions of the arms can then be deformed around the transverse bar thereby crimping the transverse bar between the arms and the face of the anchor head. This crimping secures the helical tissue-engaging element to the anchor head.

In some implementations, the tissue anchor can further include a hollow cap, adapted to be secured to the anchor head, but pins extending through the hollow cap and through a circumferential groove in the anchor head.

In some implementations, an arm deforming system is adapted to deform the transverse portions of the arms. In some implementations, the system includes a jig adapted to retain the tissue anchor such that the arms are accessible via a lumen of the helical portion. In some implementations, a pressure applicator is adapted to be inserted into the lumen of the helical portion, to engage the arms, and to apply pressure thereto, thereby to deform the arms and crimp the transverse bar between the arms and the face of the anchor head.

In accordance with some implementations, a tissue anchor includes a tissue-engaging element and/or a head. In some implementations, the tissue-engaging element can include a helical portion having a plurality of coils. In some implementations, the tissue-engaging element can include a transverse bar extending from an end of a first coil of the plurality of coils, across a chord of a circular projection of the helical portion.

In some implementations, the head can include a head portion including a driving interface and having a first diameter, and/or a bore, extending transversely through the head.

In some implementations, the transverse bar of the tissue-engaging element can extend through the bore such that the tissue-engaging element is secured to the head by the transverse bar extending through the bore.

In some implementations, the tissue anchor is sterile.

In some implementations, the chord is a diameter of the circular projection of the helical portion.

In some implementations, the head further includes a neck portion having a second diameter, smaller than the first diameter.

In some implementations, the first coil of the plurality of coils extends circumferentially around the neck portion.

In some implementations, the bore extends transversely within the head portion and the neck portion.

In some implementations, a longitudinal axis of the bore is perpendicular to a longitudinal axis of the head.

In some implementations, the tissue-engaging element is secured to the head without welding therebetween.

In some implementations, the head portion of the head includes a circumferential groove.

In some implementations, the tissue anchor further includes a cap and/or a pin. In some implementations, the cap can include a plurality of holes disposed along a circumference thereof. In some implementations, the cap can be adapted to be disposed about the head, with the pin extending through one of the plurality of holes, within the circumferential groove, and through another one of the plurality of holes, such that the cap is attached to the head portion.

In some implementations, the circumferential groove and the pin are dimensioned to allow rotation of the cap relative to the head.

In some implementations, a circumferential end of the cap extends about the head portion and obstructs motion of the transverse bar of the tissue-engaging element out of the bore.

In some implementations, the cap defines a hollow and includes a portal enabling access to the hollow.

In accordance with some implementations, a kit for manufacturing a tissue anchor includes a tissue-engaging element and/or a head. In some implementations, the tissue-engaging element can include a helical portion having a plurality of coils. In some implementations, the tissue-engaging element can include a transverse bar extending from an end of a first coil of the plurality of coils, across a chord of a circular projection of the helical portion.

In some implementations, the head can include a head portion including a driving interface and having a first diameter. In some implementations, the head can include a bore, extending transversely through the head.

In some implementations, the helical tissue-engaging element and the head portion are sterile.

In some implementations, the chord is a diameter of the circular projection of the helical portion.

In some implementations, the head further includes a neck portion having a second diameter, smaller than the first diameter.

In some implementations, the bore extends transversely within the head portion and the neck portion.

In some implementations, a longitudinal axis of the bore is perpendicular to a longitudinal axis of the head.

In some implementations, the head portion of the head includes a circumferential groove.

In some implementations, the kit further includes a pin, and/or a cap including a plurality of holes disposed along a circumference thereof. In some implementations, the cap can be adapted to be disposed about the head, with the pin extending through one of the plurality of holes, within the circumferential groove, and through another one of the plurality of holes.

In some implementations, the cap defines a hollow and includes a portal adapted to enable access to the hollow.

In accordance with some implementations, a method of manufacture of a tissue anchor, the method including obtaining a helical tissue-engaging element and obtaining a head and connecting or coupling them. In some implementations, the helical tissue-engaging element includes a helical portion having a plurality of coils. In some implementations, the helical tissue-engaging element includes a transverse bar extending from an end of a first coil of the plurality of coils.

In some implementations the transverse bar extends across a chord of a circular projection of the helical portion.

In some implementations, the head includes a head portion including a driving interface and having a first diameter. In some implementations, the head includes a bore, extending transversely through the head.

In some implementations, connecting or coupling the helical tissue-engaging element and the head includes inserting the transverse bar into the bore.

In some implementations, the method further includes sterilizing the tissue anchor.

In some implementations, the head includes a neck portion having a second diameter, smaller than the first diameter, the method further including wrapping the first coil of the helical portion circumferentially about the neck portion.

In some implementations, the method is devoid of welding.

In some implementations, the head portion of the head includes a circumferential groove.

In some implementations, the method can further include disposing a cap about the head, the cap including a plurality of holes along a circumference thereof. In some implementations, the method can further include attaching the cap to the head by placing a pin through one of the plurality of holes to another of the plurality of holes, via the circumferential groove.

In some implementations, the attaching includes attaching the cap to the head such that the cap can rotate relative to the head.

In some implementations, the attaching includes attaching the cap to the head such that a circumferential end of the cap extends about the head portion and obstructs motion of the transverse bar out of the bore.

In accordance with some implementations, a tissue anchor, including a tissue-engaging element and/or a head.

In some implementations, the tissue-engaging element can include a helical portion. In some implementations, the tissue-engaging element can include one or more of a dart, barb, hook, staple, rivet, clasp, clip, clamp, screw, etc.

In some implementations, the tissue-engaging element can include a transverse bar extending from an end of the helical portion. In some implementations the transverse bar extends across a chord of a circular projection of the helical portion.

In some implementations, the head can be attached to the tissue-engaging element. In some implementations, the head includes a driving interface, a face, and first and second arms.

In some implementations, the face can face away from the driving interface and toward the tissue-engaging element. In some implementations, the first and second arms can extend from the face.

In some implementations, each of the first and second arms can include a root portion extending away from the face; and/or a transverse portion extending from the root portion, and across the face, and terminating at an arm end. In some implementations, the root portions of the first and second arms are on opposing sides of the face, such that the arm ends of the transverse portions of the first and second arms are directed away from each other, and the transverse portions are substantially parallel to one another.

In some implementations, the head can define a gap formed between the transverse portions of the first and second arms, substantially parallel thereto. In some implementations, the transverse bar of the tissue-engaging element can extend between the face and the first and second arms, and/or can be crimped therebetween by deformation of the transverse portions about the transverse bar.

In some implementations, the tissue anchor is sterile.

In some implementations, the first and second arms are oriented in substantially opposite directions to each other.

In some implementations, the chord is a diameter of the circular projection of the helical portion.

In some implementations, a longitudinal axis of the gap is perpendicular to a longitudinal axis of the head.

In some implementations, the tissue-engaging element is secured to the head without welding therebetween.

In some implementations, the head further includes a circumferential groove.

In some implementations, the tissue anchor further includes a pin, and/or a cap including a plurality of holes disposed along a circumference thereof. In some implementations, the cap can be adapted to be disposed about the head, with the pin extending through one of the plurality of holes, within the circumferential groove, and through another one of the plurality of holes, such that the cap is attached to the head.

In some implementations, the circumferential groove and the pin are dimensioned to allow rotation of the cap relative to the head.

In some implementations, the cap defines a hollow and includes a portal enabling access to the hollow.

In accordance with some implementations, a kit for manufacture of a tissue anchor includes a helical tissue-engaging element and/or a head. In some implementations, the helical tissue-engaging element includes (i) a helical portion, and (ii) a transverse bar extending from an end of the helical portion. In some implementations, the transverse bar extends across a chord of a circular projection of the helical portion.

In some implementations, the head, includes (i) a driving interface, (ii) a face, facing away from the driving interface, and (iii) first and second arms, extending from the face.

In some implementations, each of the first and second arms include a root portion extending away from the face, and a transverse portion extending from the root portion, and across the face, each terminating at an arm end. In some implementations, the root portion of the first and second arms is on opposing sides of the face, such that the arm ends of the transverse portions of the first and second arms are directed away from each other, and the transverse portions are substantially parallel to one another.

In some implementations, a gap is formed between the transverse portions of the first and second arms, substantially parallel to the transverse portions of the first and second arms.

In some implementations, the helical tissue-engaging element and the head are sterile.

In some implementations, the chord is a diameter of the circular projection of the helical portion.

In some implementations, a longitudinal axis of the gap is perpendicular to a longitudinal axis of the head.

In some implementations, the head includes a circumferential groove.

In some implementations, the kit further includes a pin, and/or a cap including a plurality of holes disposed along a circumference thereof. In some implementations, the cap can be adapted to be disposed about the head, with the pin extending through one of the plurality of holes, within the circumferential groove, and through another one of the plurality of holes.

In some implementations, the cap defines a hollow and includes a portal adapted to enable access to the hollow.

In accordance with some implementations, a system for manufacturing a tissue anchor is provided, wherein the tissue anchor includes (i) a helical tissue-engaging element including a helical portion defining a central lumen, and a transverse bar extending from an end of the helical portion, and (ii) an anchor head having first and second arms extending from a face thereof.

In some implementations, each of the first and second arms include a root portion extending away from the face and a transverse portion extending from the root portion across the face and terminating at an arm end, the system including a jig and/or an elongate pressure applicator.

In some implementations, the jig can be adapted to receive the anchor head. In some implementations, the elongate pressure applicator can be sized and configured to extend through the central lumen of the helical portion, and adapted to apply pressure to the first and second arms, while the anchor head is held within the jig and the transverse bar extends between the face and the transverse portions of the first and second arms, to crimp the transverse bar between the transverse portions of the first and second arms and the anchor head thereby to secure the helical tissue-engaging element to the anchor head.

In some implementations, the elongate pressure applicator is adapted to apply the pressure along a vector that is colinear with a longitudinal axis of the helical portion, in a manner that crimps the transverse bar between the first and second arms and the face of the anchor head by pressing the transverse portions of the first and second arms longitudinally toward the face of the anchor head and against the transverse bar.

In some implementations, the vector is colinear with a longitudinal axis of the pressure applicator.

In accordance with some implementations, a system for manufacturing a tissue anchor includes a tissue-engaging element, a head, a jig, and/or an elongate pressure applicator. In some implementations, the tissue-engaging element can include a helical portion defining a central lumen.

In some implementations, the tissue-engaging element can include a transverse bar extending from an end of the helical portion, across a chord of a circular projection of the helical portion.

In some implementations, the head can be adapted to be attached to the tissue-engaging element. In some implementations, the head can include a driving interface, a face, facing away from the driving interface, and first and second arms.

In some implementations, the first and second arms can extend from the face, each of the first and second arms including a root portion extending away from the face, and/or a transverse portion extending from the root portion, across the face and terminating at an arm end, the root portions of the first and second arms being on opposing sides of the face, such that the arm ends of the transverse portions of the first and second arms are directed away from each other and the transverse portions are substantially parallel to one another.

In some implementations, a gap can be defined between the transverse portions of the first and second arms, substantially parallel to the transverse portions.

In some implementations, the jig can be adapted to receive the head such that the face and the first and second arms of the head are exposed during manufacture of the tissue anchor.

In some implementations, the pressure applicator can be sized and configured to extend through the central lumen of the helical portion, and to apply pressure to the first and second arms, while the head is held within the jig and the transverse bar extends between the face and the transverse portions of the first and second arms, to deform the transverse portions of the first and second arms about the transverse bar.

In some implementations, the elongate pressure applicator is adapted to apply the pressure along a vector that is colinear with a longitudinal axis of the helical portion, in a manner that crimps the transverse bar between the first and second arms and the face of the head by pressing the transverse portions of the first and second arms longitudinally toward the face of the head and against the transverse bar.

In some implementations, the vector is colinear with a longitudinal axis of the pressure applicator.

In some implementations, the head includes a circumferential groove, the system further including a pin and/or a cap including a plurality of holes disposed along a circumference thereof.

In some implementations, the cap can be adapted to be disposed about the head, with the pin extending through one of the plurality of holes, within the circumferential groove, and through another one of the plurality of holes, such that the cap is attached to the head.

In some implementations, the cap defines a hollow and includes a portal adapted to enable access to the hollow.

In accordance with some implementations, a method of manufacture of a tissue anchor, the method including obtaining a helical tissue-engaging element, obtaining a head, and connecting or coupling the helical tissue-engaging element and the head. In some implementations, the helical tissue-engaging element includes a helical portion defining a central lumen.

In some implementations, the helical tissue-engaging element includes a transverse bar extending from an end of the helical portion. In some implementations, the transverse bar extends across a chord of a circular projection of the helical portion.

In some implementations, the head includes (i) a driving interface, (ii) a face facing away from the driving interface, and (iii) first and second arms, extending from the face.

In some implementations, each of the first and second arms includes a root portion extending away from the face. In some implementations each of the first and second arms includes a transverse portion extending from the root portion, across the face, and terminating at an arm end. In some implementations, the root portions of the first and second arms are on opposing sides of the face, such that the arm ends of the transverse portions of the first and second arms are directed away from each other, and the transverse portions are substantially parallel to one another.

In some implementations, a gap is formed between the transverse portions of the first and second arms, substantially parallel thereto.

In some implementations, the method includes inserting the transverse bar into the gap.

In some implementations, the method includes rotating the helical tissue-engaging element relative to the head. In some implementations this is done such that a longitudinal axis of the transverse bar is perpendicular to a longitudinal axis of the gap and the transverse bar extends between the face and the transverse portions of the arms.

In some implementations, the method further includes applying pressure to the first and second arms to deform the transverse portions of the first and second arms about the transverse bar, thereby to secure the head to the tissue-engaging element.

In some implementations, the method is devoid of welding.

In some implementations, the method further includes, prior to applying the pressure, retaining the head within a jig, such that the first and second arms are accessible, wherein the applying pressure includes inserting an elongate pressure applicator through the central lumen of the helical portion, to apply pressure to the first and second arms, while the head is retained within the jig and the transverse bar extends between the face and the transverse portions of the first and second arms, to deform the transverse portions of the first and second arms about the transverse bar.

In some implementations, applying the pressure includes applying pressure along a vector that is colinear with a longitudinal axis of the helical portion of the tissue-engaging element.

In some implementations, applying the pressure initially crimps the transverse bar between the face and an inner surface of the first and second arms, and subsequently causes pivoting of the first and second arms to engage sides of the transverse bar.

In some implementations, the method further comprises sterilizing the tissue anchor.

In some implementations, the head includes a circumferential groove. In some implementations, the method can further include disposing a cap about the head.

In some implementations, the cap can have plurality of holes along a circumference thereof. In some implementations, the method can further include attaching the cap to the head by placing a pin through one of the plurality of holes to another of the plurality of holes, via the circumferential groove.

In some implementations, the attaching includes attaching the cap to the head such that the cap can rotate relative to the head.

In accordance with some implementations, a tissue anchor includes a tissue-engaging element and/or a head. In some implementations, the tissue-engaging element can be shaped as a helix having a plurality of coils. In some implementations, the coils can extend helically around a lumen. In some implementations the helix can be cut from a first tubular segment.

In some implementations the tissue anchor can further include a neck portion. In some implementations the neck portion can include a second tubular segment. In some implementations the neck portion can connect the head to the tissue-engaging element by being connected to the head, and extending, from the head, into the lumen.

In some implementations, the tissue anchor is sterile.

In some implementations, the neck portion is concentric with the tissue-engaging element, such that a longitudinal hollow is defined along longitudinal axes of the neck portion and the tissue-engaging element.

In some implementations, a distal end of the neck portion is frictionally held within a proximal end of the tissue-engaging element.

In some implementations, a distal end of the neck portion is soldered or adhered to a proximal end of the tissue-engaging element.

In some implementations, a distal end of the tissue-engaging element includes a sharp tip adapted to penetrate into tissue.

In some implementations, a distal end of the neck portion includes a sharp tip adapted to puncture or penetrate into tissue.

In some implementations, at least part of an external surface of the neck portion includes a recessed thread.

In some implementations, some of the plurality of coils of the helix are disposed within the recessed thread.

In some implementations, an external diameter of the second tubular segment is not greater than an internal diameter of the first tubular segment.

In some implementations, the distal end of the tissue-engaging element is adapted to transfer strain, from a surface being engaged, to the neck portion.

In some implementations, at least one of the first tubular segment and the second tubular segment includes a hypotube.

In some implementations, the helix is laser-cut from the first tubular segment.

In accordance with some implementations, a method of manufacture of a tissue anchor includes cutting a first tubular segment to form a helix. In some implementations the helix can have a plurality of coils that extend helically around a lumen. In some implementations the helix forms a tissue-engaging element.

In some implementations the method can further include connecting distal end of a second tubular segment into the lumen at a proximal end of the tissue-engaging element.

In some implementations, a proximal end of the second tubular element includes a head of the tissue anchor.

In some implementations, the method can further include attaching a proximal end of the second tubular element to a head.

In some implementations, the method can further include sterilizing the tissue anchor.

In some implementations, the cutting includes laser cutting.

In some implementations, the cutting includes cutting the first tubular segment along an entire longitudinal length thereof.

In some implementations, the connecting includes connecting the second tubular segment to the tissue-engaging element such that a longitudinal hollow is defined along longitudinal axes of the second tubular segment and of the tissue-engaging element.

In some implementations, the connecting includes frictionally connecting the distal end of the second tubular segment within the proximal end of the tissue-engaging element.

In some implementations, the connecting includes soldering or adhering the distal end of the second tubular segment to the proximal end of the tissue-engaging element.

In some implementations, at least part of an external surface of the second tubular segment includes a recessed thread, and wherein the connecting includes disposing some of the plurality of coils of the helix within the recessed thread.

In some implementations, the method can further include, prior to the connecting, etching the recessed thread into the external surface of the second tubular segment.

In some implementations, the method can further include sharpening the distal end of the tissue-engaging element to form a sharp tip adapted to penetrate into tissue.

In some implementations, the method can further include sharpening the distal end of the second tubular segment to form a sharp tip adapted to puncture or penetrate into tissue.

In accordance with some implementations, an apparatus, usable with a tissue of a subject or simulation, the apparatus including a tissue anchor. In some implementations, the tissue anchor can include a head including a notch. In some implementations, the tissue anchor can include a tissue-engaging element.

In some implementations, the tissue engaging element extends distally from the head, and is configured to be driven into the tissue by rotation of the head.

In some implementations, the apparatus can further include a tubular casing. In some implementations, the tubular casing disposed about the tissue engaging element. In some implementations, at least a portion of the tubular casing is axially compressible.

In some implementations, the apparatus can further include a locking rod. In some implementations the locking rod extends proximally (i) from a fixation point at which the locking rod is fixed to the tubular casing, and (ii) past at least the portion of the tubular casing toward the head.

In some implementations, the tissue anchor is sterile. In some implementations, the apparatus is sterile.

In some implementations, the tissue anchor can further include a neck portion connecting the head to the tissue-engaging element.

In some implementations, the tissue-engaging element includes a helical tissue engaging element.

In some implementations, the tissue anchor includes a tissue-engaging element. In some implementations, the tissue-engaging element is shaped as a helix having a plurality of coils that extend helically around a lumen. In some implementations the helix being cut from a first tubular segment.

In some implementations the tissue anchor can further include a head. In some implementations, the tissue anchor can further include a neck portion, including a second tubular segment. In some implementations the neck portion connects the head to the tissue-engaging element by being connected to the head, and extending, from the head, into the lumen.

In some implementations, the tissue anchor is rotatable relative to the tubular casing. In some implementations, the tissue-engaging element is rotatable within the tubular casing.

In some implementations, an internal diameter of the tubular casing is smaller than a diameter of the head.

In some implementations, the at least a portion includes an axially compressible spring.

In some implementations, the tubular casing is structured to resist becoming twisted during axial compression thereof.

In some implementations, the distal end of the tubular casing is textured, to create friction between the tubular casing and surface of the tissue and to prevent rotation of the tubular casing relative to the tissue during rotational driving of the tissue anchor into the tissue.

In some implementations, the locking rod is sized and configured, when the tissue anchor is driven into a tissue, to extend through the notch in the head, thereby to prevent rotation of the head relative to the tubular casing.

In some implementations, a longitudinal length of the locking rod is not greater than a longitudinal length of the tubular casing, when the tubular casing is not compressed.

In some implementations, the locking rod is disposed between an interior surface of the tubular casing and an exterior surface of the tissue-engaging element.

In some implementations, the locking rod is disposed alongside an exterior surface of the tubular casing. In some implementations, a diameter of the head is greater than a cumulative diameter of the tubular casing and the locking rod, such that the notch is adapted to be vertically aligned, in a specific rotational orientation, with the locking rod, for the locking rod to engage the notch.

In some implementations, the fixation point is adjacent a distal end of the tubular casing.

In some implementations, in a rest state of the apparatus, the tissue-engaging element extends distally out of the distal end of the tubular casing, the tubular casing is uncompressed, and the proximal end of the tubular casing is longitudinally spaced from the head.

In some implementations, during anchoring of the tissue anchor into the tissue, the tissue-engaging element extends distally out of the tubular casing and into the tissue and the tubular casing is uncompressed, until the distal end of the tubular casing engages a surface of the tissue, and the head of the tissue anchor engages the proximal end of the tubular casing.

In some implementations, upon further rotation of the tissue anchor relative to the tubular casing, the tubular casing is adapted to axially compress, to facilitate further extension of the tissue-engaging element into the tissue.

In some implementations, in an anchored and locked state of the apparatus, the tissue-engaging element extends distally out of the tubular casing and into the tissue, the distal end of the tubular casing engages the exterior surface of the tissue, the tubular casing is compressed such that a proximal end of the locking rod extends beyond the proximal end of the tubular casing and is disposed within the notch of the head, thereby preventing further rotation of the tissue anchor causing motion of the tissue anchor in a distal direction relative to the tubular casing.

In some implementations, the engagement of the locking rod within the notch facilitates rotation causing motion of the tissue anchor proximally relative to the tubular casing.

In accordance with some implementations, a kit for manufacture of an apparatus useable with a tissue of a subject or simulation can include a tissue anchor, a tubular casing, and/or a locking rod.

In some implementations, the tissue anchor can include a head including a notch. In some implementations the tissue anchor can further include a tissue-engaging element, extending distally from the head, and configured to be driven into the tissue by rotation of the head.

In some implementations, the tubular casing is adapted to be disposed about the tissue-engaging element. In some implementations, the tubular casing includes at least a portion which is axially compressible.

In some implementations, the locking rod is adapted to be fixed to the tubular casing at a fixation point and to extend proximally therefrom, past at least the portion of the tubular casing, toward the head.

In some implementations, the tissue anchor is sterile. In some implementations, the tissue anchor, the tubular casing, and the locking rod are sterile.

In some implementations, the tissue anchor can further include a neck portion connecting the head to the tissue-engaging element.

In some implementations, the tissue-engaging element includes a helical tissue engaging element.

In some implementations, the tissue anchor includes a tissue-engaging element, shaped as a helix having a plurality of coils that extend helically around a lumen, the helix being cut from a first tubular segment.

In some implementations, the tissue anchor can further include a head. In some implementations, the tissue anchor can further include a neck portion, including a second tubular segment. In some implementations, the neck portion connects the head to the tissue-engaging element by being connected to the head, and extending, from the head, into the lumen.

In some implementations, the tissue anchor is adapted to be rotatable relative to the tubular casing. In some implementations, the tissue anchor is adapted to be rotatable within the tubular casing.

In some implementations, an internal diameter of the tubular casing is smaller than a diameter of the head.

In some implementations, the at least a portion forms an axially compressible spring.

In some implementations, the tubular casing is structured to resist becoming twisted during axial compression thereof.

In some implementations, the distal end of the tubular casing is textured.

In some implementations, the locking rod is sized and configured, when the tissue anchor is driven into a tissue, to extend through the notch in the head, thereby to prevent rotation of the head relative to the tubular casing.

In some implementations, a longitudinal length of the locking rod is not greater than a longitudinal length of the tubular casing, when the tubular casing is not compressed.

In some implementations, the locking rod is adapted to be disposed between an interior surface of the tubular casing and an exterior surface of the tissue-engaging element.

In some implementations, the locking rod is adapted to be disposed alongside an exterior surface of the tubular casing.

In some implementations, a diameter of the head is greater than a cumulative diameter of the tubular casing and the locking rod, such that the notch is adapted to be vertically aligned, in a specific rotational orientation, with the locking rod, for the locking rod to engage the notch.

In accordance with some implementations, a method of manufacture of an apparatus usable with a tissue of a subject or simulation includes obtaining a tissue anchor. In some implementations, the tissue anchor includes a head including a notch and a tissue-engaging element, extending distally from the head. In some implementations, the tissue-engaging element is configured to be driven into the tissue by rotation of the head.

In some implementations, the method can further include disposing a tubular casing about the tissue-engaging element. In some implementations, at least a portion of the tubular casing is axially compressible.

In some implementations, the method can further include fixedly anchoring a locking rod to the tubular casing at a fixation point, such that the locking rod extends past at least the portion of the tubular casing toward the head.

In some implementations, the method can further include sterilizing the apparatus.

In some implementations, the obtaining of the tissue anchor includes a tissue-anchor that further includes a neck portion connecting the head to the tissue-engaging element.

In some implementations, the obtaining of the tissue anchor includes obtaining of a tissue anchor having a helical tissue engaging element.

In some implementations, the obtaining of the tissue anchor includes obtaining a tissue anchor including a tissue-engaging element. In some implementations the tissue engaging element is shaped as a helix having a plurality of coils that extend helically around a lumen. In some implementations, the helix being cut from a first tubular segment.

In some implementations, the tissue anchor can further include a head. In some implementations, the tissue anchor can further include a neck portion, including a second tubular segment. In some implementations, the neck portion connects the head to the tissue-engaging element by being connected to the head, and extending, from the head, into the lumen.

In some implementations, the obtaining of the tissue anchor includes manufacturing the tissue anchor.

In some implementations, the manufacturing of the tissue anchor includes cutting a first tubular segment to form a helix having a plurality of coils that extend helically around a lumen, thereby to form a tissue-engaging element. In some implementations, the manufacturing can further include connecting distal end of a second tubular segment into the lumen at a proximal end of the tissue-engaging element.

In some implementations, the disposing of the tubular casing includes disposing the tubular casing such that the tissue anchor is rotatable relative to the tubular casing.

In some implementations, the disposing of the tubular casing includes disposing the tubular casing such that the tissue anchor is rotatable within the tubular casing.

In some implementations, the fixedly anchoring of the locking rod includes fixedly anchoring the locking rod to an interior surface of the tubular casing, between the tubular casing and the tissue-engaging element.

In some implementations, the fixedly anchoring of the locking rod includes fixedly anchoring the locking rod to an exterior surface of the tubular casing, such that the locking rod extends longitudinally alongside the tubular casing.

In some implementations, the fixedly anchoring includes fixedly anchoring the locking rod to the tubular casing at the fixation point, the fixation point being adjacent a distal end of the tubular casing.

Any of the above systems, assemblies, devices, apparatuses, components, etc. can be sterilized (e.g., with heat, radiation, ethylene oxide, hydrogen peroxide, etc.) to ensure they are safe for use with patients, and the methods herein can comprise (or additional methods comprise or consist of) sterilization of one or more systems, devices, apparatuses, components, etc. herein (e.g., with heat, radiation, ethylene oxide, hydrogen peroxide, etc.).

The present invention will be more fully understood from the following detailed description of examples thereof, taken together with the drawings, in which:

### BRIEF DESCRIPTION OF THE DRAWINGS

The foregoing discussion will be understood more readily from the following detailed description when taken in conjunction with the accompanying Figures, in which:

FIG. 1 is an exploded perspective view illustration of a tissue anchor, in accordance with some implementations;

FIG. 2 is an illustration of the tissue anchor of FIG. 1, when constructed, in perspective and sectional views;

FIG. 3 is a perspective view illustration of the tissue anchor of FIG. 2, having a cap removed therefrom, in accordance with some implementations;

FIG. 4 is a sectional illustration of the tissue anchor of FIG. 3, taken along section lines IV-IV in FIG. 3;

FIGS. 5A and 5B are perspective illustrations of a head of a tissue anchor, in accordance with some implementations;

FIGS. 6A and 6B are sectional illustrations of the head of the tissue anchor of FIGS. 5A and 5B;

FIGS. 7A and 7B are perspective view illustrations of a tissue anchor including the head of FIGS. 5A to 6B, in accordance with some implementations;

FIGS. 8A, 8B, 8C, 8D, and 8E are perspective and sectional view illustrations at different stages of manufacture of the tissue anchor of FIGS. 7A and 7B;

FIG. 9 is an exploded perspective view illustration of an example tissue anchor, in accordance with some implementations;

FIG. 10 is a perspective view illustration of the tissue anchor of FIG. 9, when constructed;

FIG. 11 is an exploded perspective view illustration of an example tissue anchor, in accordance with some implementations;

FIGS. 12A and 12B are, respectively, a perspective view illustration and a sectional illustration of the tissue anchor of FIG. 11, when constructed;

FIGS. 13A, 13B, 13C, and 13D are schematic illustrations at different stages of use of the tissue anchors of FIGS. 9 to 12B;

FIG. 14 is an exploded perspective view illustration of an example apparatus including a tissue anchor, in accordance with some implementations;

FIG. 15 is a perspective view illustration and sectional view illustration of the apparatus of FIG. 14, when constructed;

FIGS. 16A, 16B, and 16C are sectional illustrations at different stages of use of the apparatus of FIGS. 14 to 15;

FIG. 17 is a schematic illustration of an example structure of an axially compressible portion of the apparatus of FIGS. 14 to 16C, at different stages of compression thereof, in accordance with some implementations;

FIG. 18 is a perspective view illustration and sectional view illustration of an example apparatus including a tissue anchor in accordance with some implementations; and

FIG. 19 is a perspective view illustration of an example apparatus including a tissue anchor in accordance with some implementations.

### DETAILED DESCRIPTION

The principles of the tissue anchors may be better understood with reference to the drawings and the following description.

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features can be omitted or simplified in order not to obscure the disclosure. Additionally, in order to avoid undue clutter from having too many reference numbers and lead lines on a particular drawing, some elements may not be explicitly identified in every drawing that contains that element.

It is to be understood that the scope of the invention is not limited in its application to the details of construction and the arrangement of the components set forth in the following description or illustrated in the drawings. The invention is capable of other implementations or of being practiced or carried out in various ways. Furthermore, it is to be understood that the phraseology and terminology employed in the disclosure is for the purpose of description and should not be regarded as limiting.

For the purposes of this application, the term “subject” relates to any mammal, particularly humans.

For the purposes of this application, the term “cardiac tissue” relates to any tissue of the heart, and includes, for example, any wall of the heart and any tissue of any heart valve.

Various implementations and examples herein relate to tissue anchors for implantation in tissue of a subject or simulation, and to methods of manufacture thereof, particularly methods of manufacture that reduce a requirement for, and/or dependence on, welding.

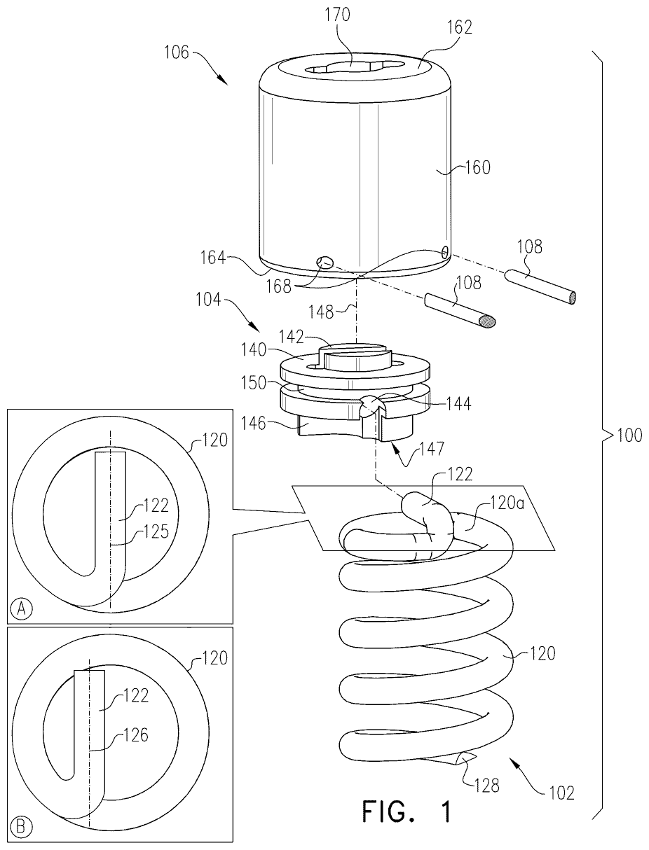

Referring now to the drawings, an example tissue anchor 100 according to some implementations is shown in FIGS. 1 in an exploded perspective view.

As seen in FIG. 1, in some implementations, tissue anchor 100 includes a helical tissue-engaging element 102, and anchor head 104. In some implementations, tissue anchor 100 further includes a cap 106, adapted to be held by one or more pins 108, as explained hereinbelow.

While a helical tissue-engaging element is often described herein for illustrative purposes, the concepts herein are not limited to helical tissue-engaging elements and other types of tissue-engaging elements can be used (e.g., comprising darts, staples, rivets, hooks, clips, clasps, clamps, barbs, etc.).

In some implementations, helical tissue-engaging element 102 includes a helical portion 120 having a plurality of coils, a first coil of which is labeled by reference numeral 120a.

In some implementations, a transverse bar 122 extends from an end of first coil 120a, across helical portion 120. More specifically, transverse bar 122 extends across (e.g., traverses) a circular projection of helical portion 120, e.g., across an end of a lumen defined by the helical portion. In some implementations, such as that shown in inset A of FIG. 1, which is a top plan view of helical tissue-engaging element 102, transverse bar 122 extends across a diameter 125 of the circular projection. However, in some implementations, transverse bar 122 can extend across a non-diameter chord of the circular projection, such as chord 126 schematically shown in inset B of FIG. 1. That is, insets A and B illustrate variants of way in which transverse bar 122 extends across the circular projection of helical portion 120 (e.g., across the end of the lumen defined by the helical portion).

In some implementations, helical portion 120 can include a sharp point 128, at an opposite end to first coil 120a.

In some implementations, anchor head 104 includes a head portion 140, including a driving interface 142. In some implementations, a bore 144 extends transversely through the head. In some implementations, the head portion 140 has a first diameter.

In some implementations, anchor head 104 further includes a neck portion 146, having a second diameter, the second diameter being smaller than the first diameter.

In some implementations, head 104 can have a face 147 that faces away from driving interface 142 and/or toward helical portion 120. Thus, upon anchoring of anchor 100, face 147 may face, or be pressed against, the tissue into which helical portion 120 is driven. As shown, face 147 may be defined by (e.g., may be a surface of) neck portion 146.

In some implementations, a longitudinal axis of bore 144 is perpendicular to a longitudinal axis 148 of anchor head 104. In some implementations, bore 144 extends transversely only within head portion 140. In some implementations, bore 144 extends transversely within head portion 140 and through neck portion 146.

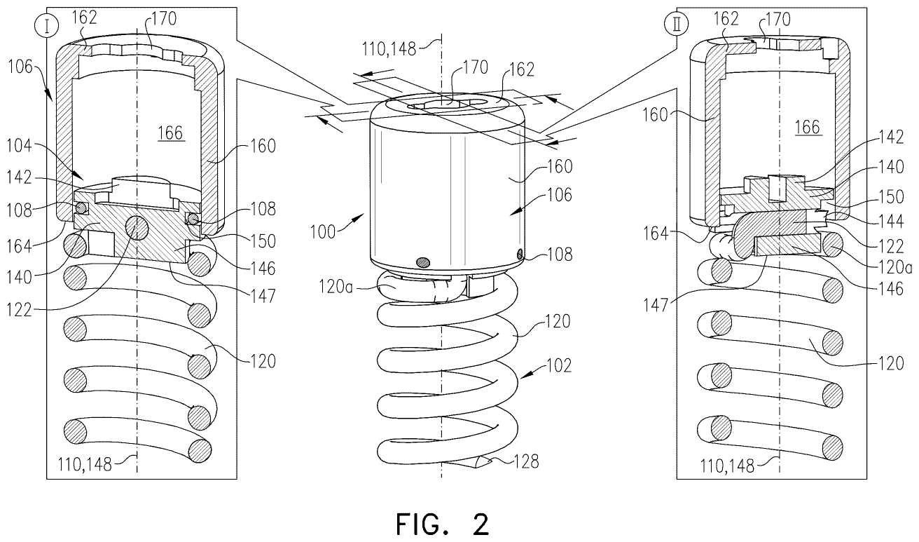

As shown in FIG. 2, longitudinal axis 148 can be colinear or coincident with an anchor axis 110 of anchor 100, once the anchor is assembled. Anchor axis 110 can be defined by tissue-engaging element 102, e.g., by the tissue-engaging element being configured to be driven into tissue along the anchor axis, and/or by the anchor axis being the helix axis of helical portion 120.

In some implementations, helical portion 120 and transverse bar 122 can be formed from a single continuous piece of stock material (e.g., stock wire) that is shaped to define both the helical portion and the transverse bar.

In some implementations, head portion 140 includes a circumferential groove 150, which can be laterally open. Circumferential groove 150 is adapted for attachment of anchor head 104 to cap 106, as explained hereinbelow.

In some implementations, cap 106 has a substantially tubular body 160 terminating at one end at a surface 162, and at the opposing end in a lip 164, such that the cap defines a hollow 166 (see FIG. 2). As explained in further detail hereinbelow, hollow 166 is adapted to receive at least part of head portion 140.

In some implementations, multiple holes 168 are formed in tubular body 160, for example adjacent lip 164 thereof, and are adapted to accommodate pin(s) 108, as explained herein. Holes 168 can be provided is pairs, with each pin 108 extending through both bores of the pair, e.g., with each end of the pin disposed in a respective bore. The two holes 168 of each pair can be axially aligned with each other, e.g., as shown. The example illustrated has four holes 168 accommodating two pins 108, but other configurations are possible, such as two bores (e.g., with one pin), six bores (e.g., with three pins), or eight bores (e.g., with four pins).

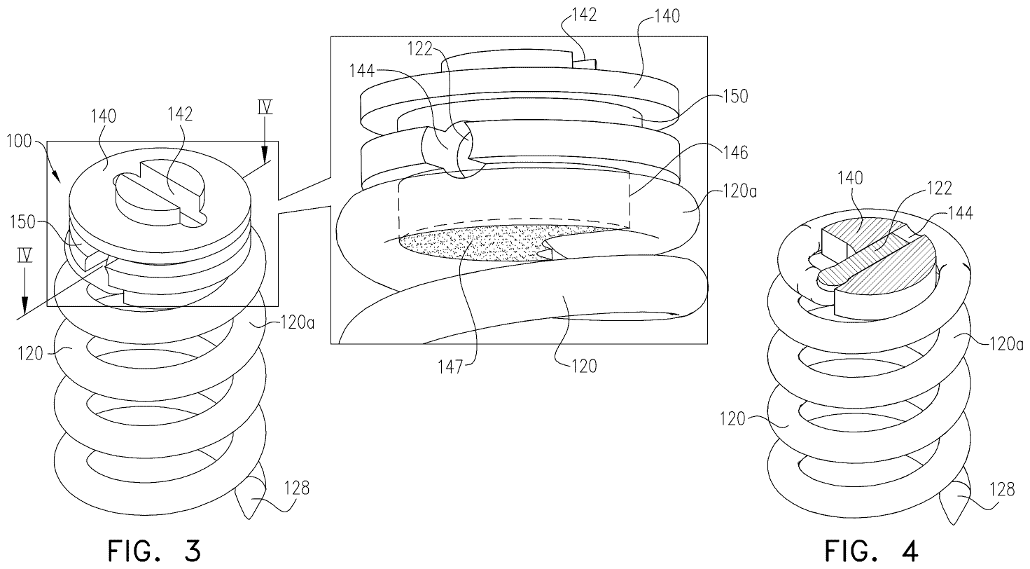

Reference is additionally made to FIG. 2, which is an illustration of the tissue anchor 100, when constructed, in perspective and sectional views, to FIG. 3, which is a perspective view illustration of the tissue anchor 100 having cap 106 removed therefrom, and to FIG. 4, which is a sectional illustration of the tissue anchor 100 of FIG. 3.

As seen, for construction of tissue anchor 100, transverse bar 122 of tissue-engaging element 102 is inserted into bore 144 of anchor head 104, as seen clearly in sectional frames I and II of FIG. 2 and in FIG. 4. Transverse bar 122 extending through bore 144 secures tissue-engaging element 102 to head 104. This securing may advantageously require no welding.

In some implementations, first coil 120a of tissue-engaging element 102 is wrapped circumferentially about neck portion 146 of anchor head 104, as seen clearly in sectional frame II of FIG. 2. In some implementations this wrapping secures transverse bar 122 within bore 144 (e.g., inhibits the transverse bar from exiting the bore), thereby further securing tissue-engaging element 102 to head 104. In some implementations this arrangement can be achieved by first coil 120a sliding over face 117 as transverse bar 122 is advanced into bore 144, and then snapping into place on the far side of neck portion 146 as the transverse bar reaches full insertion into the bore (i.e., the side opposite the entry point of the transverse bar into the bore).

In some implementations, e.g., implementations in which anchor 100 includes cap 106, the cap can be attached to anchor head 104 by disposing the tubular body 160 about the anchor head 104, such that the anchor head is disposed in hollow 166, and placing pin(s) 108 to extend from one hole 168 to another hole 168, via circumferential groove 150. The presence of pin(s) 108 in groove 150 inhibits cap 106 from being lifted off of anchor head 104, i.e., prevents axial movement of the cap away from the anchor head. However, in some implementations, pin(s) 108 and circumferential groove 150 can be dimensioned to allow rotation of cap 106 relative to head 104, e.g., the pins can be sufficiently loose within the groove.

As seen clearly in sectional frame II of FIG. 2, in some implementations, lip 164 of cap 106 extends about head portion 140 and obstructs motion of transverse bar 122 of tissue-engaging element 102 out of bore 144.

In some implementations, surface 162 of cap 106 includes a portal 170 that provides access to hollow 166, e.g., access, by an anchor driver, to driving interface 142 disposed within the hollow. In some implementations hollow 166 can house an additional component, e.g., one or more components of an implant of which anchor 100 forms a part. For example, hollow 166 can house a winch configured to tension a tether of an implant of which anchor 100 forms a part. Portal 170 can provide access to such an additional component. For example, for implementations in which hollow 166 houses a winch, portal 170 can provide access to the winch by a tool that actuates the winch.

In some implementations, tissue-engaging element 102 and anchor head 104 can be provided as part of a kit for manufacturing tissue anchor 100, e.g., with the head not yet attached to the tissue-engaging element. In some implementations, the kit can further include cap 106 and pin(s) 108.

It is to be appreciated that all the connections between tissue-engaging element 102, anchor head 104, cap 106, and pin(s) 108 can be based on frictional and mechanical forces, and may advantageously require no welding. For example, transverse bar 122 can have an interference fit (e.g., a press fit or a driving fit) in bore 144, and/or pin(s) 108 can have an interference fit in holes 168.

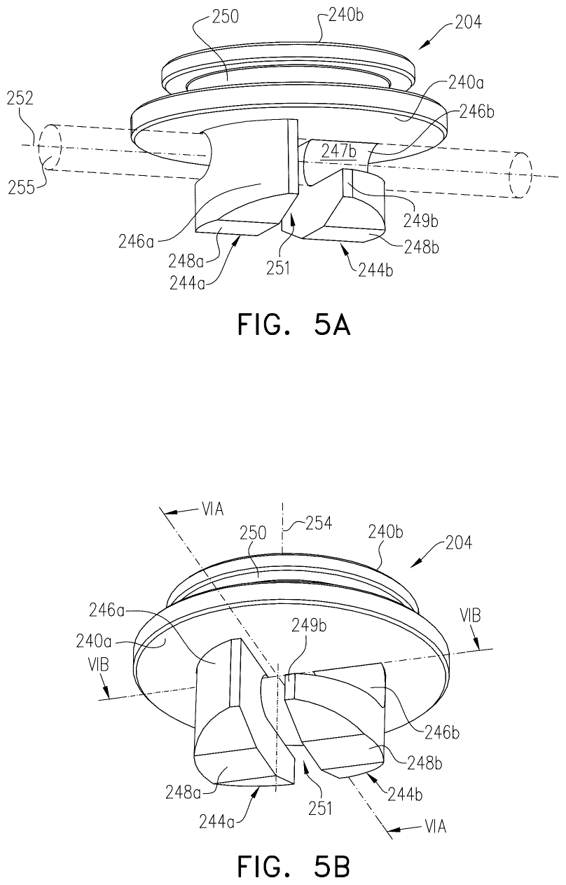

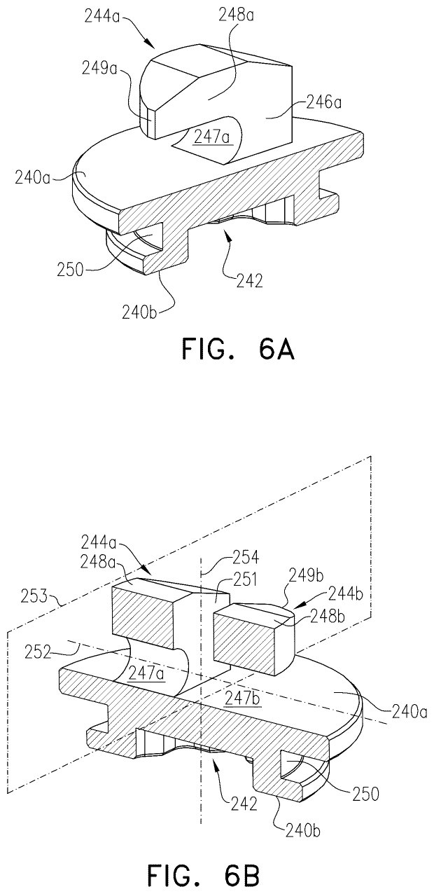

Reference is now made to FIGS. 5A and 5B, which are schematic perspective illustrations of an anchor head 204 of a tissue anchor 200, in accordance with some implementations, and to FIGS. 6A and 6B, which are schematic sectional illustrations of the anchor head 204.

Anchor head 204 defines a first face 240a, and a second face 240b. First face 240a is adapted to face toward a tissue-engaging element of the tissue anchor, as explained herein. Anchor head 204 can include a driving interface 242 via which an anchor driver can engage the anchor head and apply an anchoring force (e.g., torque) in order to drive the tissue anchor into tissue.

A first arm 244a and a second arm 244b each extends from first face 240a. First arm 244a has a root portion 246a and a transverse portion 248a terminating at an arm end 249a. Second arm 244b has a root portion 246b and a transverse portion 248b terminating at an arm end 249b. For each arm, the root portion extends away from face 240a, and the transverse portion extends, from the root portion, across the face, e.g., substantially parallel to the face. Thus, the transverse portion is attached to head 204 via the root portion.

Arms 244a and 244b are disposed on either side of a midline (e.g., a diameter) of face 240a. The cross-section of FIG. 6A lies on this midline (see also the corresponding cross-section indicator VIA in FIG. 5B). As shown, transverse portions 248a and 248b extend alongside the midline (e.g., parallel to the midline and to each other), defining a gap 251 therebetween, the gap lying on a gap plane 253 on which the midline lies. Root portions 246a and 246b of the arms can also be disposed on the same side of gap 251 as their corresponding transverse portions and, as shown, in some implementations are not disposed on any midline (e.g., diameter) of face 240a.

As shown, arms 244a and 244b can be oriented in substantially opposite directions to each other, e.g., with root portion 246a substantially opposite arm end 249b, and root portion 246b substantially opposite arm end 249a. For example, and as shown, the arrangement of the arms can have 2-fold (i.e., 180 degree) rotational symmetry but not reflectional symmetry.

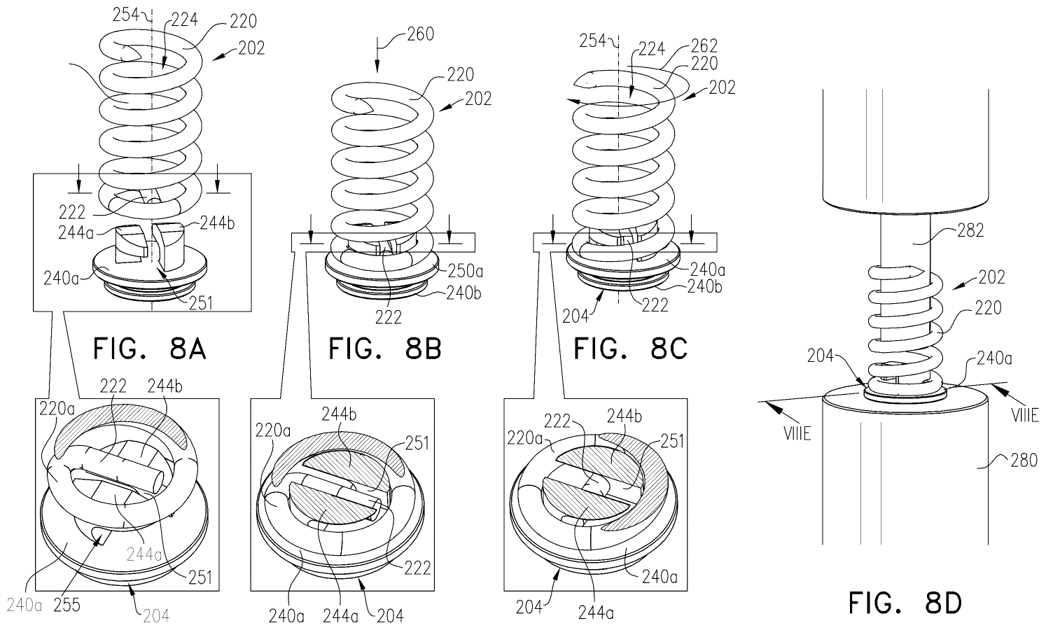

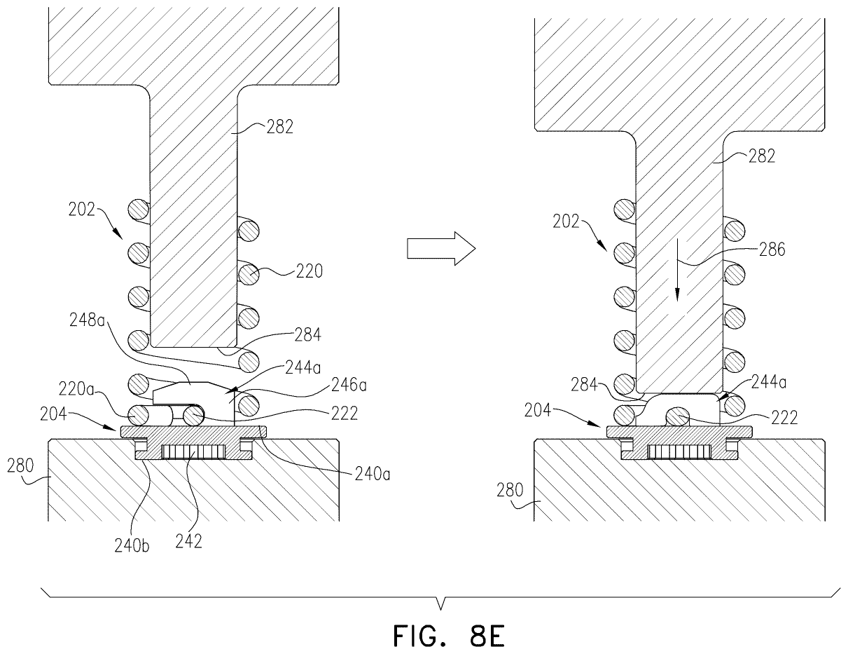

As explained in further detail hereinbelow with respect to FIGS. 8A to 8E, gap 251 is sized and configured to receive a transverse bar of a tissue-engaging element which is adapted to be connected to anchor head 204 to form the tissue anchor.

A cavity 247a is defined between the transverse portion 248a of first arm 244a and face 240a, the cavity being limited, on one side thereof, by root portion 246a. Similarly, a cavity 247b is defined between the transverse portion 248b of first arm 244b and face 240a, the cavity being limited, on one side thereof, by root portion 246b.

In some implementations, second face 240b has a first diameter, and first face 240a has a second diameter, the second diameter being smaller than the first diameter.

In some implementations, a longitudinal bore 255 is described by cavities 247a and 247b collectively. That is, bore 255 is not a closed continuous bore, but is a “virtual” bore, e.g., an artifact of the shape and alignment of the arms and their cavities. FIG. 5A includes a projection of bore 255 which, in the particular, example shown is circular in cross-section. In some implementations, a longitudinal axis 252 of bore 255 is perpendicular to a longitudinal axis 254 of anchor head 204 and/or to gap plane 253.

In some implementations, anchor head 204 includes a circumferential groove 250. Circumferential groove 250 is adapted for attachment of anchor head 204 to a cap using pins, e.g., substantially as described hereinabove with respect to FIGS. 1-4 and with respect to anchor head 104 and cap 106. That is, anchor 200 can include such a cap.

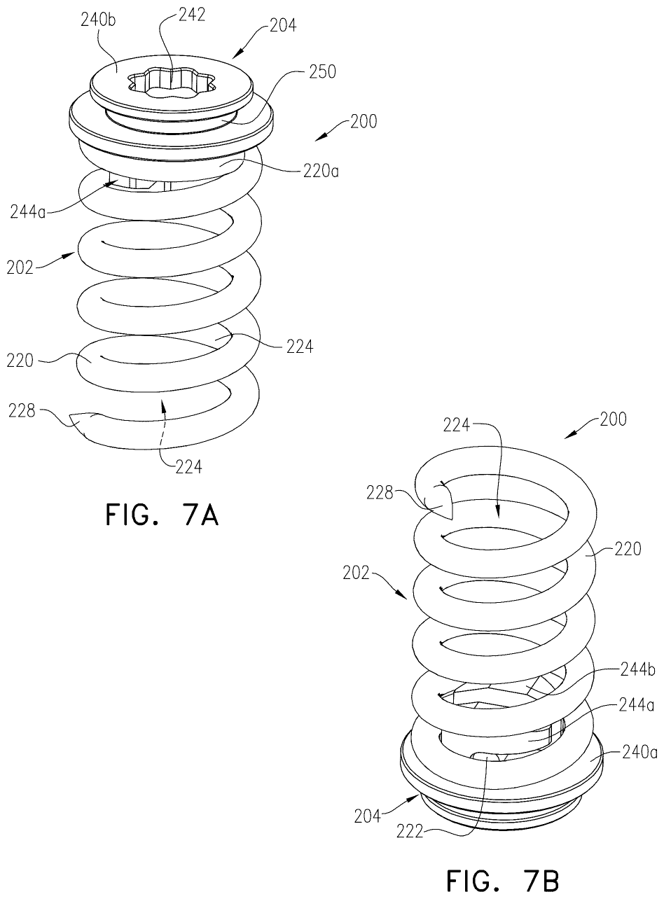

FIGS. 7A and 7B are perspective view illustrations of tissue anchor 200 including anchor head 204 of FIGS. 5A to 6B, in accordance with some implementations.

In some implementations, tissue anchor 200 includes, in addition to anchor head 204, a helical tissue-engaging element 202 (though other non-helical tissue-engagement elements can be used).

In some implementations, helical tissue-engaging element 202 includes a helical portion 220 having a plurality of coils, a first coil of which is labeled by reference numeral 220a.

In some implementations, a transverse bar 222 extends from an end of first coil 220a, across helical portion 220. More specifically, transverse bar 222 extends across a chord of a circular projection of helical portion 220, as explained in detail hereinabove with respect to FIG. 1.

In some implementations, helical portion 220 defines a central lumen 224 by circumscribing the central lumen. Central lumen 224 can be colinear with axis 254. In some implementations, helical portion 220 can have a sharp point 228, at an opposite end to first coil 220a.

In some implementations, helical portion 220 and transverse bar 222 can be formed from a single continuous piece of stock material (e.g., stock wire) that is shaped to define both the helical portion and the transverse bar. Such stock wire can have a thickness of 0.2-1 mm, such as 0.4-0.7 mm. Examples of materials from which such stock wire can be formed include stainless steel, titanium, and/or cobalt chrome.

Anchor head 204 (e.g., arms 244a and 244b) can comprise (e.g., can be formed from) stainless steel, titanium, and/or cobalt chrome. In some implementations, the material from which the arms are formed is softer and/or more malleable than the material from which tissue-engaging element 202 is formed.

In some implementations, transverse bar 222 of tissue-engaging element 202 extends through bore 255 between face 240a and transverse portions 248a and 248b of respective arms 244a and 244b. In some implementations, tissue-engaging element 202 is secured to head 204 by deformation (e.g., crimping) of transverse portions 248a and 248b against and about transverse bar 222, as explained in further detail hereinbelow with respect to FIGS. 8A-8E.

Reference is now additionally made to FIGS. 8A, 8B, 8C, 8D, and 8E, which are perspective and sectional view illustrations of various stages of construction and/or manufacture of tissue anchor 200 of FIGS. 7A and 7B.

As seen in FIG. 8A, initially, tissue-engaging element 202 is disposed adjacent anchor head 204, such first face 240a of the anchor head faces toward first coil 220a of the tissue-engaging element. Additionally, transverse bar 222 is aligned with gap 251 of anchor head 204.

In FIG. 8B, tissue-engaging element 202 is moved vertically relative to anchor head 204, e.g., in the direction of arrow 260, such that transverse bar 222 enters gap 251. Gap 251 may be only slightly (e.g., no more than 20 percent, such as no more than 10 percent, such as no more than 5 percent) wider than transverse bar 222. In some implementations, the tissue-engaging element is moved relative to the anchor head until transverse bar 222 contacts first face 240a of anchor head 204.

As seen in FIG. 8C, tissue-engaging element 202 is then rotated (e.g., about axis 254) relative to anchor head 204, for example in the direction of arrow 262. This rotation positions transverse bar 222 within cavities 247a and 247b. The cavities may be only slightly taller than transverse bar 222, e.g., such that the transverse bar fits snugly within the cavities. This rotation may be until transverse bar 222 abuts the root portions of the arms, and/or is disposed through bore 255 (which is labeled in the inset of FIG. 8A). In some implementations, this rotation is 90 degrees of rotation.

Turning to FIG. 8D, it is seen that tissue anchor 200 is then stabilized, e.g., by placing the tissue anchor (e.g., anchor head 204 thereof) within a hollow of a jig 280. In this arrangement, first face 240a is accessible, via lumen 224 of tissue-engaging element 202.

As also seen in FIG. 8D, as well as in the left frame of FIG. 8E, an elongate pressure applicator 282, terminating at a press surface 284, is sized and configured to extend through central lumen 224 of helical portion 220 of tissue-engaging element 202.

As seen in the right frame of FIG. 8E, pressure applicator 282 is moved toward first face 240a in the direction of arrow 286, while head 204 is retained within jig 280. When press surface 284 of pressure applicator 282 contacts arms 244a and 244b, it applies pressure to the arms, e.g., along a vector that is colinear with the longitudinal axis of pressure applicator 282 and/or the longitudinal axis of helical portion 220. The pressure applied to arms 244a and 244b deforms transverse portions 248a and 248b of the arms about transverse bar 222, thereby crimping the transverse bar between the arms and the face of anchor head 204, and securing tissue-engaging element 202 to the anchor head.

This deformation can exploit the malleability and/or compliance of arms 244a and 244b. Moreover, this deformation can involve not merely cantilever-like bending of the transverse portions of the arms over the transverse bar, but can include more general malleation (e.g., squashing) of the arms to conform to the shape of the transverse bar and/or face 240a. In some implementations, this effect is facilitated by the transverse portion of each arm initially being thicker at a mid-region of the transverse portion (below which transverse bar 222 is disposed) than at the arm end, and/or by the mid-region of the transverse portion initially being further from face 240a and therefore being the first part of the arm, against which press surface 284 presses. This malleation may advantageously provide more stable fixation between tissue-engaging element 202 and anchor head 204 compared to merely folding the arms over the transverse bar. For example, it may enhance both (i) sandwiching of transverse bar 222 between the mid-portion of the transverse portions of the arms, and (ii) wrapping of the transverse portions about the transverse bar.

In some implementations, tissue-engaging element 202 and anchor head 204 can be provided as part of a kit for manufacturing tissue anchor 200. In some implementations, the kit can further include a cap and one or more pins, similar to cap 106 and pins 108 of FIGS. 1-4. In some implementations, the kit can further include jig 280 and pressure applicator 282. In some implementations, anchor head 204 can be provided alone, e.g., for use with a tissue-engaging element such as tissue-engaging element 202.

It is to be appreciated that the connection between tissue-engaging element 202 and anchor head 204 may require no welding.

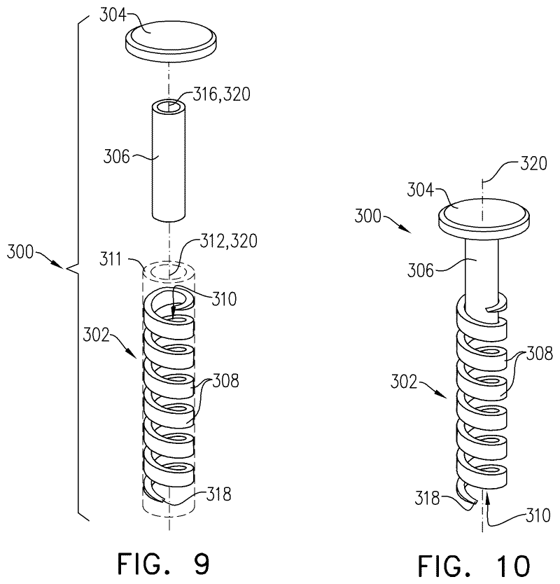

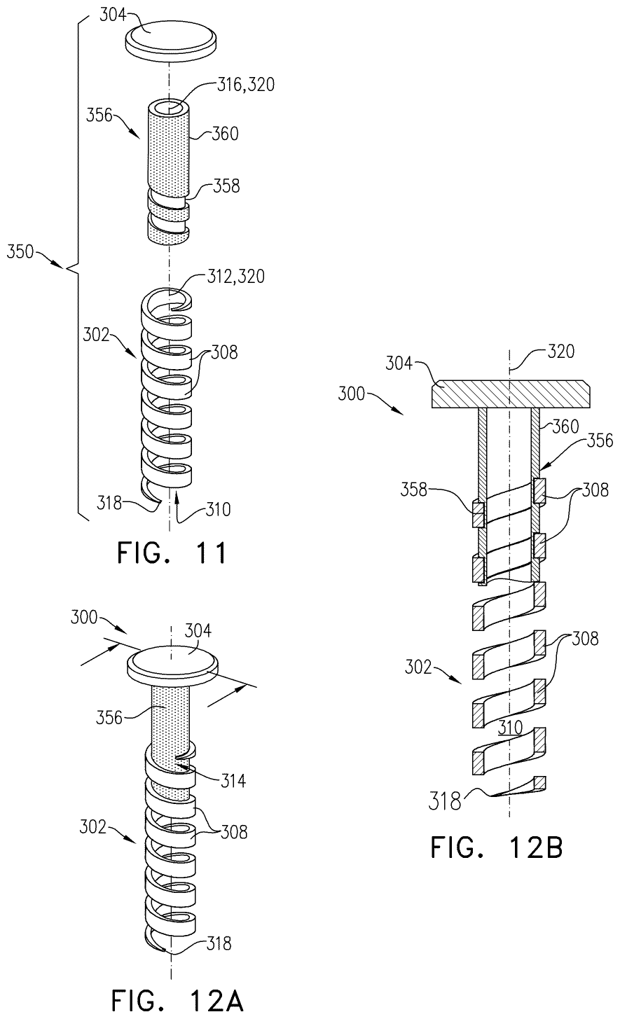

Reference is now made to FIG. 9, which is an exploded perspective view illustration of an example tissue anchor 300, in accordance with some implementations, and to FIG. 10, which is a perspective view illustration of tissue anchor 300, when constructed.

As seen in FIG. 9, tissue anchor 300 includes a tissue-engaging element 302, a head 304, and a neck portion 306.

In some implementations, tissue-engaging element 302 is shaped as a helix having a plurality of coils 308, which extend helically around a lumen 310. The helix (e.g., the entirety of tissue-engaging element 302) can be cut from a first tubular segment 311, for example by laser-cutting, or by any other suitable cutting method. For example, first tubular segment 311 can be a piece of stock tubing, e.g., comprising a metal such as stainless steel or cobalt-chrome. In some implementations, the tissue-engaging element does not include a helix.

Head 304 is illustrated as a simple disc. However, head 304 can include various interfaces for interaction of tissue anchor 300 with other devices. In some implementations, head 304 can include a driving interface (for example similar to driving interface 142 described hereinabove with respect to FIG. 1). In some implementations, head 304 can include an eyelet for threading of a line or tether (e.g., a suture or a wire) therethrough, for example as described in U.S. Patent Application Publication 2021/0145584 to Kasher et al., which is incorporated herein by reference.

In some implementations, neck portion 306 is cylindrical. In some implementations, neck portion 306 can be tubular, e.g., can include or can be a second tubular segment. For example, neck portion 306 (e.g., the tubular segment thereof) can be a piece of stock tubing, e.g., comprising a metal such as stainless steel or cobalt chrome.

For manufacture of anchor 300, the helix is cut from first tubular segment 311 to form tissue-engaging element 302, and the distal end of neck portion 306 is inserted into lumen 310 at the proximal end of tissue-engaging element 302, such that neck portion 306 extends from head 304 into lumen 310. In some implementations, an external diameter of neck portion 306 is no greater than an internal diameter of tissue-engaging element 302 (e.g., than the diameter of lumen 310).

In some implementations, at least part of head 304 can be integrally formed with neck portion 306. For example, a piece of stock tubing with a thick sidewall can have its outer diameter reduced (e.g., by a cutting technique such as turning) at neck portion 306, leaving a greater outer diameter at head 304.

In some implementations, head 304 can be separate from neck portion 306, and manufacture of tissue anchor 300 can further include attaching head 304 to neck portion 306, e.g., by welding and/or mating shaped surfaces.

In some implementations, a longitudinal axis 316 of neck portion 306 can be colinear or coincident a longitudinal axis 312 of tissue-engaging element 302 once the anchor is assembled, e.g., thereby defining a central longitudinal axis 320 of the anchor. In some implementations, a longitudinal hollow can be defined along longitudinal axes 312 and 316, from a distal end of head 304 to a distal end of tissue-engaging element 302.

In some implementations, first tubular segment 311 comprises a hypotube. In some implementations, the second tubular segment forming neck portion 306 comprises a hypotube.

In some implementations, neck portion 306 and tissue-engaging element 302 can be dimensioned to fit snugly together. In some implementations, once the distal end of neck portion 306 is disposed within lumen 310, it is held there frictionally (e.g., an interference fit), e.g., without additional adhesion such as an adhesive, welding, or brazing. Alternatively or additionally, the distal end of neck portion 306 can be adhered, welded, or brazed within lumen 310, e.g., with or without an interference fit.

In some implementations, a distal end of tissue-engaging element 302 comprises a sharp tip 318, adapted to penetrate into tissue.

In some implementations, a distal end of neck portion 306 can be sharpened, or can include a sharp tip, adapted to puncture tissue or to penetrate into tissue.

In some implementations, neck portion 306 defines multiple small holes (e.g., pores) therewithin to promote tissue regrowth around tissue anchor 300 once the anchor is implanted within the tissue.

In some implementations, a kit for manufacture of tissue anchor 300 can include tissue-engaging element 302, head 304, and neck portion 306, such that manufacture of tissue anchor 300 only requires connection of the components of the tissue anchor.

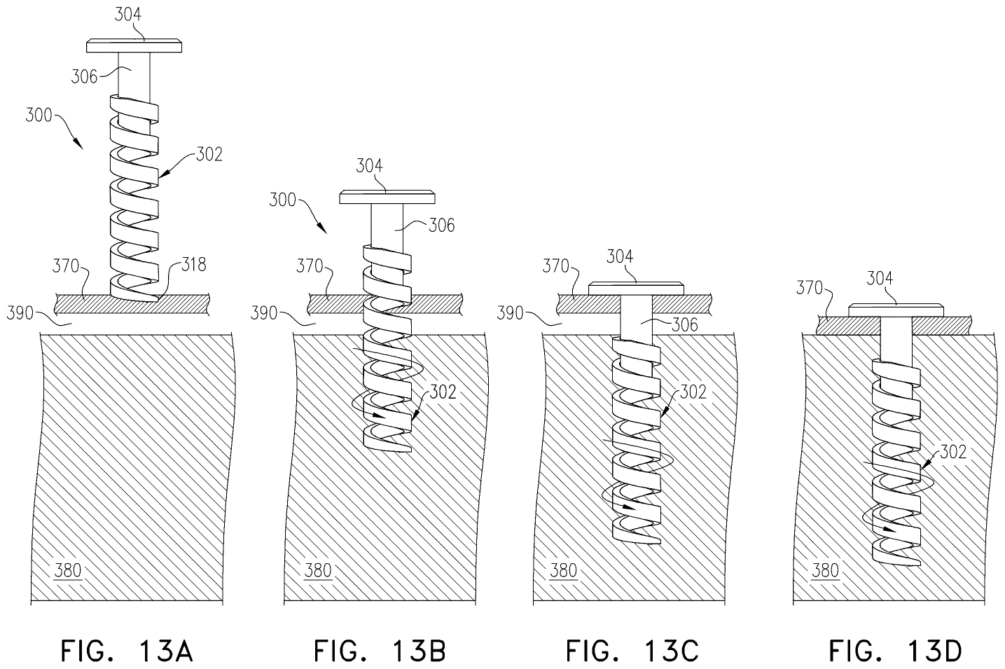

Reference is now made to FIG. 11, which is an exploded perspective view illustration of a tissue anchor 350, in accordance with some implementations, and to FIGS. 12A and 12B, which are, respectively, a perspective view illustration and a sectional illustration of tissue anchor 350, when constructed.

In some implementations, tissue anchor 350 is substantially similar to tissue anchor 300, and similarly includes tissue-engaging element 302, and head 304, substantially as described hereinabove with respect to FIGS. 9 and 10.

Instead of neck portion 306 of tissue anchor 300, tissue anchor 350 includes a neck portion 356, having a recessed external thread 358 extending along at least part of an external surface 360 of the neck portion.

In some implementations, neck portion 356 can be formed by etching, cutting (e.g., turning), or otherwise forming, thread 358 in a tubular segment, similar to the second tubular segment forming neck portion 306 of tissue anchor 300 described hereinabove.

As clearly seen in FIGS. 12A and 12B, in tissue anchor 350, some of coils 308 of tissue-engaging element 302 are disposed within recessed thread 358. Thus, in some implementations, an external diameter of neck portion 356 is greater than an internal diameter of tissue-engaging element 302 (e.g., than the diameter of lumen 310).

In some implementations, manufacture of tissue anchor 350 is similar to manufacture of tissue anchor 300 described hereinabove. Specifically, manufacture of tissue anchor 350 can include forming recessed thread 358 in external surface 360 of a tubular segment, to form neck portion 356. In some implementations, connecting of neck portion 356 into lumen 310 of tissue-engaging element 302 can include threading (e.g., screwing) thread 358 into some of coils 308.

In some implementations, neck portion 356 and tissue-engaging element 302 can be dimensioned to fit snugly together, e.g., with coils 308 fitting snugly within recessed thread 358. In some implementations, once the distal end of neck portion 356 is disposed within lumen 310, it is held there frictionally (e.g., an interference fit), e.g., without additional adhesion such as an adhesive, welding, or brazing. Alternatively or additionally, an adhesive, welding, or brazing can be used, e.g., with or without an interference fit.

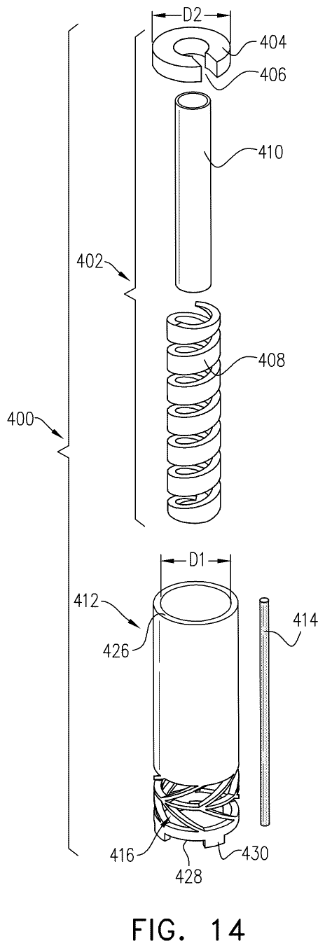

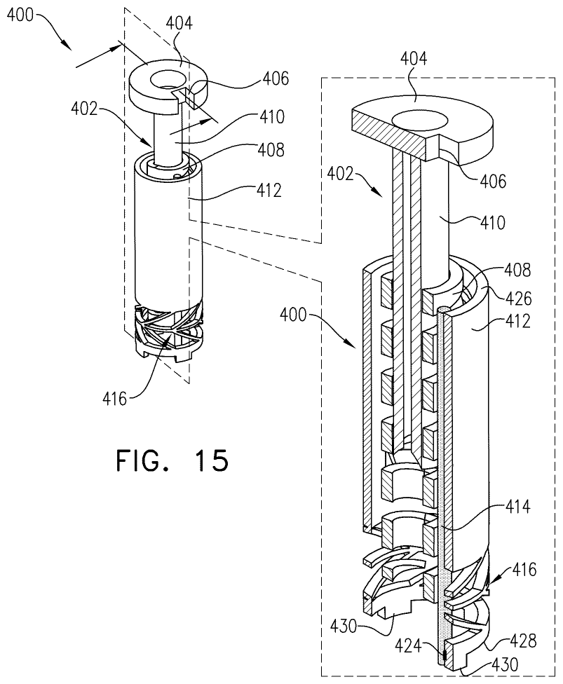

FIGS. 13A, 13B, 13C, and 13D are schematic illustrations at different stages of use of tissue anchors 300 and 350 to anchor a device 370 to tissue 380. The reference numerals shown in FIGS. 13A to 13D are those of tissue anchor 300, but it is to be appreciated that the disclosure is similarly applicable to tissue anchor 350.

In some implementations, device 370 can be, for example, an implant, e.g., a sheet and/or sleeve thereof. In some implementations, the sheet and/or sleeve can comprise a fabric and/or a film, which can comprise a polymer and/or a silicone. Such an implant comprising a sheet and/or a sleeve can be as described in any of the following publications, each of which is incorporated herein by reference for all purposes:
U.S. patent application Ser. No. 14/437,373 to Sheps et al., filed Apr. 21, 2015, which published as US 2015/0272734 (now U.S. Pat. No. 9,949,828);
U.S. patent application Ser. No. 15/782,687 to Iflah et al., filed Oct. 12, 2017, which published as US 2018/0049875 (now U.S. Pat. No. 10,765,514);
International Patent Application PCT/US2021/039587 to Chau et al., filed Jun. 29, 2021, which published as WO 2022/006087;
International Patent Application PCT/US2022/052834 to Chau et al., filed Dec. 14, 2022.

That is, anchor 300 or anchor 350 can be used in place of one or more of the anchors in any of the above publications.

In some implementations, anchor 300 is driven (e.g., by an anchor driver that applies force (e.g., torque) to head 304) such that tip 318 of tissue-engaging element 302 engages, and punctures, device 370 (FIG. 13A). In the particular situation shown, a gap 390 exists between device 370 and tissue 380. As tissue anchor 300 is driven further, tissue engaging element extends through device 370 and into tissue 380 (FIG. 13B). At this stage, gap 390 remains, e.g., because tissue-engaging element 302 moves at the same axial rate through both device 370 and tissue 380 as it is rotated.

In FIG. 13C, tissue anchor 300 has been driven further, such that (i) tissue-engaging element 302 has left device 370 (e.g., with the material of the device exiting a proximal gap 314 at the proximal-most coil 308 of the tissue-engaging element), and (ii) neck 356 extends through the device. Gap 390 still remains but, unlike tissue-engaging element 302, neck portion 306 can rotate freely within device 370. Thus, further driving of tissue anchor 300 drives tissue-engaging element 302 further axially into tissue 380 but not further axially with respect to device 370, thereby advantageously reducing (e.g., eliminating) gap 390 without undesirably twisting or applying torque to the device (FIG. 13D). Neck portion 306 can remain extended through device 370 in the anchored state of tissue anchor 300.

One or more components of tissue anchor 300 being cut from stock tubing can confer on the tissue anchor manufacturing advantages such as increased simplicity, efficiency, and/or consistency. For example, manufacturing tissue-engaging element 308 by cutting it from a piece of stock tubing having known properties may be simpler and more efficient than forming a helical tissue-engaging element by bending a wire and sharpening its tip-especially if the wire must be bent to form an axial neck in addition to a helix. Furthermore, the connection between tissue-engaging element 308 and neck 356 may be simpler and more efficient to form, and/or may be stronger and/or more durable, than other techniques such as welding a head to a wire-formed tissue-engaging element.

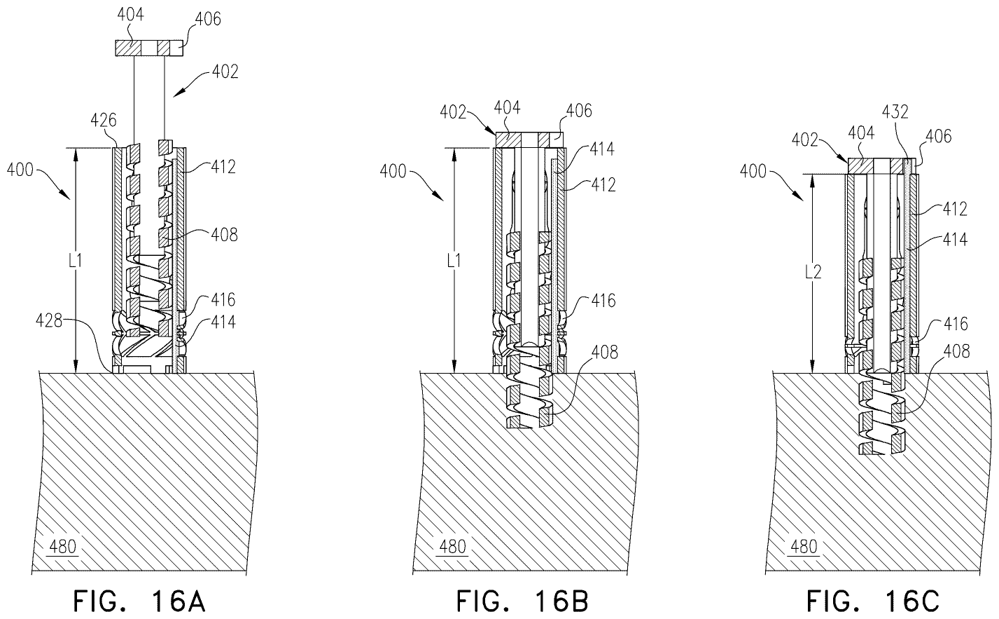

Reference is now made to FIG. 14, which is an exploded perspective view illustration of an apparatus 400 including a tissue anchor 402, in accordance with some implementations, and to FIG. 15, which is a perspective view illustration and sectional view illustration of the apparatus 400, when constructed.

Tissue anchor 402 includes a head 404 including a notch 406, and a tissue engaging element 408. Tissue-engaging element extends distally from the head and is configured to be driven into the tissue by rotation of the head.

In some implementations, tissue anchor 402 further includes a neck portion 410, connecting head 404 to tissue-engaging element 408.

In some implementations, tissue-engaging element 408 is, or includes, a helical tissue engaging element.

Tissue anchor 402 may be any suitable tissue anchor, including tissue anchors known in the art. In some implementations, tissue anchor 402 is a tissue anchor as described hereinabove with respect to FIGS. 1-4. In some implementations, tissue anchor 402 is a tissue anchor as described hereinabove with respect to FIGS. 5A to 8E. In some implementations, such as the illustrated example, tissue anchor 402 is a tissue anchor as described hereinabove with respect to FIGS. 9 and 10. In some implementations, tissue anchor 402 is a tissue anchor as described hereinabove with respect to FIGS. 11 to 12B.

In some implementations, head 404 can include, in addition to notch 406, various interfaces for interaction of tissue anchor 402 with other devices. In some implementations, head 404 can include a driving interface (for example similar to driving interface 142 described hereinabove with respect to FIG. 1). In some implementations, head 404 can include an eyelet for threading of a suture or wire therethrough, for example as described in in U.S. Patent Application Publication 2021/0145584 to Kasher et al., which is incorporated herein by reference.

In some implementations, apparatus 400 further includes a tubular casing 412 and/or a locking rod 414. At least a portion of tubular casing 412 is axially compressible.

For example, in the illustrated example, tubular casing 412 includes an axially compressible portion 416, adjacent a distal end thereof. However, it is to be appreciated that axially compressible portion 416 can be located at a different longitudinal location along tubular casing 412, and can span a different longitudinal portion of the tubular casing. In some implementations, axially compressible portion 416 can cover the majority, or even the entirety, of the tubular length of tubular casing 412.

In some implementations, axially compressible portion 416 comprises an axially compressible spring. An example of a suitable axially compressible spring is shown in FIG. 17, at different stages of compression thereof.

As shown clearly in FIG. 15, tubular casing 412 is disposed about tissue-engaging element 408 of tissue anchor 402. Locking rod 414 extends from a fixation point 424, at which the locking rod is fixed to tubular casing 412, and past at least axially compressible portion 416, toward head 404 of tissue anchor 402.

As explained in further detail hereinbelow, locking rod 414 is sized and configured, when tissue anchor 402 is driven into a tissue, to extend through notch 406 in head 404, and to prevent rotation, in at least one direction, of tissue anchor 402 relative to tubular casing 412, e.g., preventing overtightening.

In some implementations, and as shown, a longitudinal length of locking rod 414 is not greater than a longitudinal length of tubular casing 412, when the tubular casing is not compressed.

In some implementations, and as shown, locking rod 414 is disposed between the interior surface of the tubular casing and an exterior surface of tissue-engaging element 408.

In some implementations, such as in the example illustrated in FIGS. 14 and 15, fixation point 424 is at or adjacent a distal end of tubular casing 412. In some implementations, a length of locking rod 414 can be slightly shorter than a length of tubular casing 412.

In some implementations, tissue anchor 402 is rotatable relative to tubular casing 412, with tissue-engaging element 408 being rotatable within tubular casing 412.

In some implementations, an internal diameter D1 of tubular casing 412 is smaller than a diameter D2 of head 404. As such, when tissue anchor 402 is advanced, in a distal direction, within tubular casing 412, head 404 eventually contacts a proximal surface 426 of tubular casing 412.

In some implementations, a distal surface 428 of tubular casing 412 can be configured to grip a surface of tissue during use of apparatus 400, thereby preventing rotation of tubular casing 412 relative to the tissue during rotational driving of tissue anchor 402 into the tissue, as explained in further detail hereinbelow. This configuration may be provided, for example, by texturing (e.g., knurling) or, as shown, by including protrusions 430 or barbs.

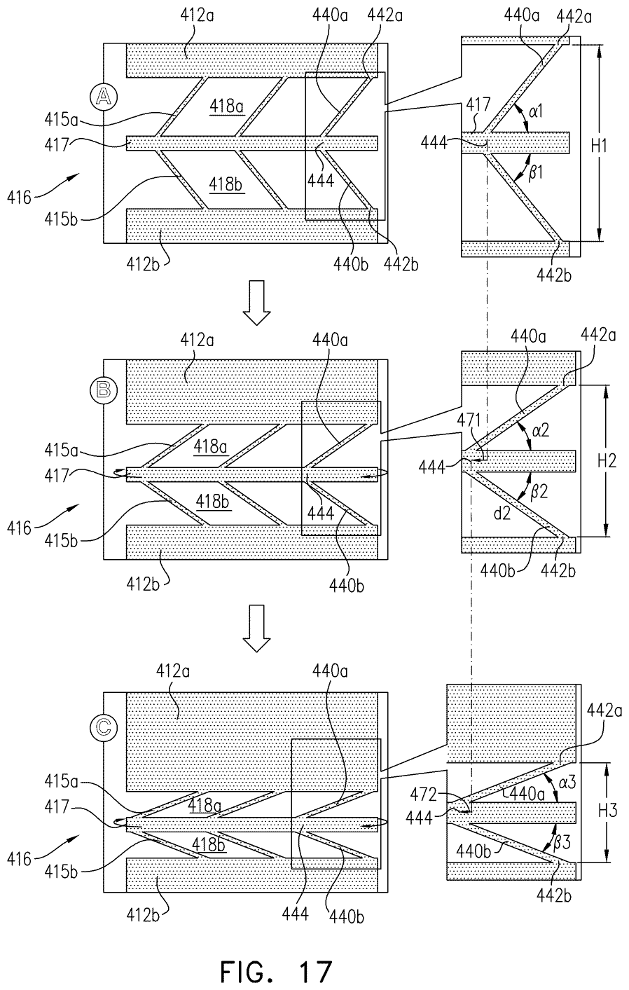

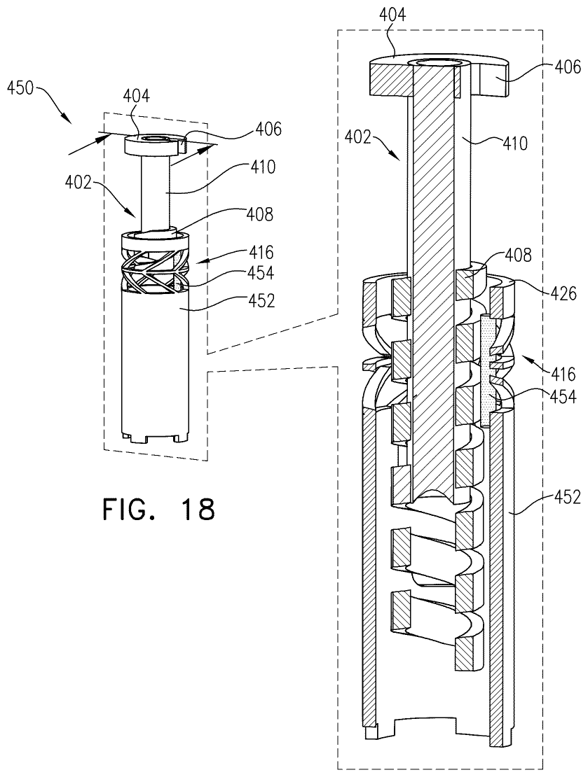

Reference is now additionally made to FIGS. 16A, 16B, and 16C, which are sectional illustrations at different stages of use of apparatus 400.

Specifically, FIG. 16A illustrates a rest state of apparatus 400, when the apparatus is placed adjacent tissue 480, but no other operation has been carried out thereon. In the rest state, tissue-engaging element 408 is disposed within tubular casing 412, such that head 404 is longitudinally spaced proximally from proximal surface 426 of the tubular casing. In the rest state of apparatus 400, axially-compressible portion 416 is uncompressed, and tubular casing 412 has a length L1.

In some implementations, a distal end of tissue-engaging element 408 can extend distally out of tubular casing 412 while apparatus 400 is in its rest state.

FIG. 16B illustrates a state of apparatus 400 following subsequent driving of tissue anchor 402, such that tissue-engaging element 408 begins to advance distally out of the tubular casing and screws into the tissue. For illustrative purposes, FIG. 16B shows this driving to have occurred until a point that head 404 contacts (e.g., abuts) proximal surface 426 of tubular casing 412. At this stage, portion 416 remains uncompressed, and tubular casing 412 still has length L1.

In some implementations, additional driving (e.g., screwing-in) of tissue anchor 402 causes head 404 to press against proximal surface 426 (e.g., rotationally sliding against the surface), thereby pressing tubular casing 412 against tissue 480. This pressing causes tubular casing 412 to axially shorten via compression of portion 416. Eventually, casing 412 reaches a longitudinal length L2 (which is shorter than length L1) at which a proximal end 432 of locking rod 414 extends proximally beyond proximal surface 426 of the tubular casing and into notch 406 (FIG. 16C). This rotationally locks tissue anchor 402 to tubular casing 412. This locking prevents further rotation of tissue anchor 402 with respect to tubular casing 412 and, optionally combined with gripping of the tissue by distal surface 428 of the tubular casing, thereby reduces a likelihood of undesirably overtightening the tissue anchor, e.g., that may otherwise tear the tissue.

Once apparatus 400 is thus locked, axially-compressible portion is typically locked in its compressed state.

In some implementations, engagement of locking rod 414 within notch 406 prevents both clockwise and anticlockwise rotation of tissue anchor 402 relative to tubular casing 412.

In some implementations, engagement of locking rod 414 within notch 406 prevents rotation of tissue anchor 402 relative to tubular casing 412 in the screwing-in direction, but allows rotation in a reverse, unscrewing direction, e.g., facilitating removal of tissue anchor 402 from tissue 480 by reverse rotation of the tissue anchor relative to the tubular casing.

Reference is now made to FIG. 17, which is a schematic illustration of an example structure of axially compressible portion 416, at different stages of compression thereof, in accordance with some implementations.

In the illustrated example, axially compressible portion 416 divides tubular casing 412 into a proximal portion 412a, disposed proximally to portion 416, and a distal portion 412b, disposed distally to portion 416.

In the example shown, axially compressible portion 416 comprises first and second rows of cells (e.g., cutout segments), the cells of each row being separated from each other, and/or at least partly defined, by struts 415, e.g., struts 415a of the first row and struts 415b of the second row. In some implementations, the rows are separated from each other by a band 417. Each of the rows, and the band, can be circumferential. Cells 418a in the first row are parallelograms oriented in a first circumferential direction, and cells 418b in the second row form parallelograms oriented in a second, opposing circumferential direction. For example, and as shown, the first and second rows of cells can exhibit substantial reflectional symmetry across band 417. This arrangement can alternatively be described with reference to struts 415. For example, struts 415a lie in a first oblique orientation with respect to (and/or extend in a first helical direction around) a central longitudinal axis of casing 412, and struts 415b lie in an opposite such oblique orientation (and/or extend in an opposite such helical direction).

Enlarged portion A shows compressible portion 416 when it is not compressed, and has a length H1, e.g., in a resting state of tubular casing 412.

Description of the compression of axially compressible portion 416 is provided herein with respect to two specific struts, indicated by reference numerals 440a and 440b. strut 440a is connected to proximal portion 412a at point 442a, and strut 440b is connected to distal portion 412b at point 442b. In some implementations, and as shown, struts 440a and 440b intersect at a point 444, on band 417. Acute angles a are formed between strut 440a and proximal portion 412a, and/or between strut 440a and band 417. Acute angles B are formed between strut 440b and distal portion 412b, and/or between strut 440b and band 417.

Enlarged portions B and C show progressive stages of compression of axially compressible portion 416 from a length H1 (enlarged portion A), to progressively shorter lengths H2 (enlarged portion B) and H3 (enlarged portion C). As seen, upon application of axial force to tubular casing 412 and to axially compressible portion 416, cells 418a and 418b compress by deflection of struts 415, which causes band 417 to rotate relative to proximal portion 412a and to distal portion 412b, e.g., as indicated by left-pointing arrows.

Turning specifically to struts 440a and 440b, it is seen that as axial force is applied to axially compressible portion 416, struts 440a and 440b deflect causing the size of acute angles a and B to decrease, e.g., compare the inset of enlarged portion A with the inset of enlarged portion B. Responsively, band 417 rotates relative to both proximal portion 412a and distal portion 412b, e.g., as indicated by an arrow 471 in the inset of enlarged portion B. Similarly, comparison of enlarged portions B and C demonstrates that struts 440a and 440b have further deflected, and band 417 has rotated further clockwise relative to its placement in enlarged portion B, e.g., as indicated by an arrow 472 in the inset of enlarged portion C. For the sake of illustration, arrows 471 and 472 indicate changes in the rotational position of a point 444 between compressive states—but it is to be understood that this is arbitrary, and that any given point on band 417 will move similarly.

It is to be noted that the structure of compressible portion 416 advantageously allows casing 412 to be axially compressed while maintaining rotational alignment between portions 412a and 412b (e.g., the portions do not rotate relative to each other). This is visible in FIG. 17 by observing that points 442a move axially closer to points 442b without moving rotationally relative to points 442b. Furthermore, axially compressible portion 416 retains its exterior diameter during compression, e.g., such that, advantageously, little or no bulging occurs during axial compression.

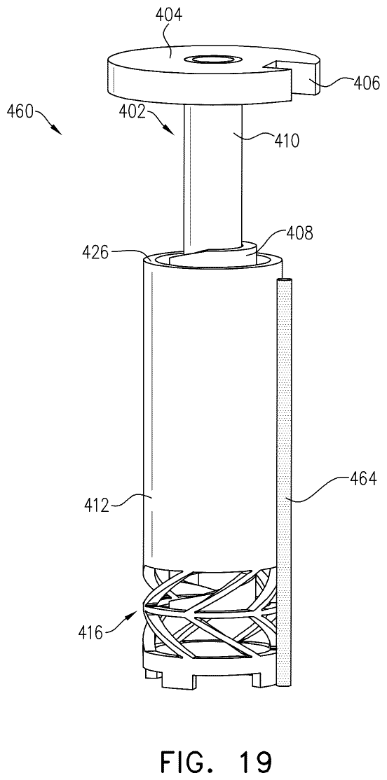

Reference is now made to FIG. 18, which is a perspective view illustration and sectional view illustration of an apparatus 450, including a tissue anchor 402.

Apparatus 450 is substantially similar to apparatus 400, and similarly includes tissue anchor 402 and its components, substantially as described hereinabove with respect to FIGS. 14 to 16C, and functions in a similar manner.

In apparatus 450, a tubular casing 452 replacing tubular casing 412 of apparatus 400. Tubular casing 452 differs from tubular casing 412 in that the axially compressible portion 416 is located adjacent the proximal end of the tubular casing, rather than adjacent the distal end thereof. Additionally, locking rod 414 of FIGS. 14 to 15 is replaced with a locking rod 454.

As seen in FIG. 18, when the axially compressible portion 416 is disposed adjacent the proximal end of the tubular casing, locking rod 454 can be fixed to the tubular casing at any point along the length of the casing, provided that the fixation point is distal to the compressible portion. Consequently, locking rod 454 can be shorter, and need not be present in all of the distal part of the casing. In the illustrated example, locking rod 454 is fixed to tubular casing 452 just distally of compressible portion 416. Additionally, a short locking rod 454 may be sufficient for accomplishing the functionality of rod 414 as described with respect to FIGS. 16A to 16C.

Reference is now made to FIG. 19, which is a perspective view illustration of an apparatus 460, including a tissue anchor 402.

In some implementations, apparatus 460 is substantially similar to apparatus 400, and similarly includes tissue anchor 402 and its components, substantially as described hereinabove with respect to FIGS. 14 to 16C, and functions in a similar manner.

In some implementations, in apparatus 460, locking rod 464 is fixed to an exterior surface of tubular casing 412, and functions in a similar manner to locking rod 414 of FIGS. 14 to 16C. The ability to position locking rod 464 to the exterior of tubular casing 412 is facilitated by the non-bulging nature of the compressible portion of the tubular casing, as described hereinabove.

In some implementations, when locking rod 464 is exterior to tubular casing 412, a diameter of head 404 may be sufficiently large that, in at least some rotational orientations of the tissue anchor, locking rod 464 can be aligned with notch 406, as shown in FIG. 19.

Reference is again made to FIGS. 1-19. The manufacturing techniques described herein, and/or the tissue anchors manufactured therewith, can be used, mutatis mutandis, in combination with one or more of the implants, delivery tools, and/or techniques described one or more of the following references, each of which is incorporated herein by reference. For example, the anchors described in the following references can be manufactured using the techniques described herein:
U.S. patent application Ser. No. 14/437,373 to Sheps et al., which published as US 2015/0272734;
U.S. patent application Ser. No. 15/782,687 to Iflah et al., which published as US 2018/0049875;
International Patent Application PCT/IB2020/060044 to Kasher et al., which published as WO 2021/084407;
International Patent Application PCT/IB2021/058665 to Halabi et al., which published as WO 2022/064401;
International Patent Application PCT/IB2022/051099 to Shafigh et al., which published as WO 2022/172149;
International Patent Application PCT/;US2021/039587 to Chau et al., which published as WO 2022/006087
International Patent Application PCT/IB2021/060436 to Tennenbaum et al., which published as WO 2022/101817.

It should be understood that the use of “and/or” is defined inclusively such that the term “a and/or b” should be read to include the sets: “a and b,” “a or b,” “a,” “b.”

### Example Applications (Some Non-Limiting Examples of the Concepts Herein are Recited Below):

Example 1. A tissue anchor, comprising: (A) a helical tissue-engaging element, including: (i) a helical portion having a plurality of coils, (e.g., the coils may extend helically around a lumen defined by the helical portion); and/or (ii) a transverse bar extending from an end of a first coil of the plurality of coils, across a chord of a circular projection of the helical portion (e.g., transversely across an end of the lumen); and/or (B) a head, comprising: (i) a head portion including a driving interface and having a first diameter; and/or (ii) a bore, extending transversely through the head, wherein the transverse bar of the tissue-engaging element extends through the bore, and the tissue-engaging element is secured to the head by the transverse bar extending through the bore.

Example 2. The tissue anchor according to example 1, wherein the tissue anchor is sterile.

Example 3. The tissue anchor according to any one of examples 1 to 2, wherein the chord is a diameter of the circular projection of the helical portion.

Example 4. The tissue anchor according to any one of examples 1 to 3, wherein the head further comprises a neck portion having a second diameter, smaller than the first diameter.

Example 5. The tissue anchor according to example 4, wherein the first coil of the plurality of coils extends circumferentially around the neck portion.

Example 6. The tissue anchor according to any one of examples 4 to 5, wherein the bore extends transversely within the head portion and the neck portion.

Example 7. The tissue anchor according to any one of examples 1 to 6, wherein a longitudinal axis of the bore is perpendicular to a longitudinal axis of the head.

Example 8. The tissue anchor according to any one of examples 1 to 7, wherein the tissue-engaging element is secured to the head without welding therebetween.

Example 9. The tissue anchor according to any one of examples 1 to 8, wherein the head portion of the head includes a circumferential groove.

Example 10. The tissue anchor according to example 9, further comprising: (A) a cap including a plurality of holes disposed along a circumference thereof; and/or (B) a pin, wherein the cap is adapted to be disposed about the head, with the pin extending through one of the plurality of holes, within the circumferential groove, and through another one of the plurality of holes, such that the cap is attached to the head portion.

Example 11. The tissue anchor according to example 10, wherein the circumferential groove and the pin are dimensioned to allow rotation of the cap relative to the head.

Example 12. The tissue anchor according to any one of examples 10 to 11, wherein a circumferential end of the cap extends about the head portion and obstructs motion of the transverse bar of the tissue-engaging element out of the bore.

Example 13. The tissue anchor according to any one of examples 10 to 12, wherein the cap defines a hollow and includes a portal enabling access to the hollow.

Example 14. A kit for manufacturing a tissue anchor, the kit comprising: (A) a helical tissue-engaging element, including: (i) a helical portion having a plurality of coils (e.g., that may extend helically around a lumen defined by the helical portion); and/or (ii) a transverse bar extending from an end of a first coil of the plurality of coils, across a chord of a circular projection of the helical portion (e.g., transversely across an end of the lumen); and/or (B) a head, comprising: (i) a head portion including a driving interface and having a first diameter; and/or (ii) a bore, extending transversely through the head.

Example 15. The kit according to example 14, wherein the helical tissue-engaging element and the head portion are sterile.

Example 16. The kit according to any one of examples 14 to 15, wherein the chord is a diameter of the circular projection of the helical portion.

Example 17. The kit according to any one of examples 14 to 16, wherein the head further comprises a neck portion having a second diameter, smaller than the first diameter.

Example 18. The kit according to example 17, wherein the bore extends transversely within the head portion and the neck portion.

Example 19. The kit according to any one of examples 14 to 18, wherein a longitudinal axis of the bore is perpendicular to a longitudinal axis of the head.

Example 20. The kit according to any one of examples 14 to 19, wherein the head portion of the head includes a circumferential groove.

Example 21. The kit according to example 20, further comprising: (A) a cap including a plurality of holes disposed along a circumference thereof; and/or (B) a pin, wherein the cap is adapted to be disposed about the head, with the pin extending through one of the plurality of holes, within the circumferential groove, and through another one of the plurality of holes.

Example 22. The kit according to example 21, wherein the cap defines a hollow and includes a portal adapted to enable access to the hollow.

Example 23. A method of manufacture of a tissue anchor, the method comprising: (a) obtaining a helical tissue-engaging element, including: (i) a helical portion having a plurality of coils (e.g., that extend helically around a lumen defined by the helical portion); and/or (ii) a transverse bar extending from an end of a first coil of the plurality of coils, across a chord of a circular projection of the helical portion (e.g., traversing across an end of the lumen); (b) obtaining a head, comprising: (i) a head portion including a driving interface and having a first diameter; and/or (ii) a bore, extending transversely through the head; and/or (c) inserting the transverse bar into the bore (e.g., affixing the head to the tissue-engaging element by inserting the transverse bar into the bore).

Example 24. The method according to example 23, further comprising sterilizing the tissue anchor.

Example 25. The method according to any one of examples 23 to 24,wherein the head comprises a neck portion having a second diameter, smaller than the first diameter, the method further comprising: (d) wrapping the first coil of the helical portion circumferentially about the neck portion (e.g., affixing the head to the tissue-engaging element may comprise wrapping the first coil of the helical portion circumferentially about the neck portion.)

Example 26. The method according to any one of examples 23 to 25, wherein the method is devoid of welding.

Example 27. The method according to any one of examples 23 to 26, wherein the head portion of the head includes a circumferential groove, the method further comprising: (e) disposing a cap about the head, the cap including a plurality of holes along a circumference thereof; and/or (f) attaching the cap to the head by placing a pin through one of the plurality of holes to another of the plurality of holes, via the circumferential groove.

Example 28. The method according to example 27, wherein the attaching comprises attaching the cap to the head such that the cap can rotate relative to the head.

Example 29. The method according to any one of example 27 or example 28, wherein the attaching comprises attaching the cap to the head such that a circumferential end of the cap extends about the head portion and obstructs motion of the transverse bar out of the bore.

Example 30. A tissue anchor, comprising: (a) a helical tissue-engaging element, including: (i) a helical portion; and/or (ii) a transverse bar extending from an end of the helical portion, across a chord of a circular projection of the helical portion; and/or (b) a head, attached to the tissue-engaging element, the head comprising: (i) a driving interface; (ii) a face, facing away from the driving interface and toward the tissue-engaging element; (iii) first and second arms, extending from the face, each of the first and second arms including: (A) a root portion extending away from the face; and/or (B) a transverse portion extending from the root portion, and across the face, and terminating at an arm end, the root portions of the first and second arms being on opposing sides of the face, such that the arm ends of the transverse portions of the first and second arms are directed away from each other and the transverse portions are substantially parallel to one another; and/or (iv) a gap formed between the transverse portions of the first and second arms, substantially parallel thereto, wherein the transverse bar of the tissue-engaging element extends between the face and the first and second arms, and is crimped therebetween by deformation of the transverse portions about the transverse bar.

Example 31. The tissue anchor according to example 30, wherein the tissue anchor is sterile.

Example 32. The tissue anchor according to any one of examples 30 to 31, wherein the first and second arms are oriented in substantially opposite directions to each other.

Example 33. The tissue anchor according to any one of examples 30 to 32, wherein the chord is a diameter of the circular projection of the helical portion.

Example 34. The tissue anchor according to any one of examples 30 to 33, wherein a longitudinal axis of the gap is perpendicular to a longitudinal axis of the head.

Example 35. The tissue anchor according to any one of examples 30 to 34, wherein the tissue-engaging element is secured to the head without welding therebetween.

Example 36. The tissue anchor according to any one of examples 30 to 35, wherein the head further includes a circumferential groove.

Example 37. The tissue anchor according to example 36, further comprising: (i) a cap including a plurality of holes disposed along a circumference thereof; and/or (ii) a pin, wherein the cap is adapted to be disposed about the head, with the pin extending through one of the plurality of holes, within the circumferential groove, and through another one of the plurality of holes, such that the cap is attached to the head.

Example 38. The tissue anchor according to example 37, wherein the circumferential groove and the pin are dimensioned to allow rotation of the cap relative to the head.

Example 39. The tissue anchor according to any one of examples 37 to 38, wherein the cap defines a hollow and includes a portal enabling access to the hollow.

Example 40. A kit for manufacture of a tissue anchor, the kit comprising: (a) a helical tissue-engaging element, including: (i) a helical portion; and/or (ii) a transverse bar extending from an end of the helical portion, across a chord of a circular projection of the helical portion; and/or (b) a head, comprising: (i) a driving interface; (ii) a face, facing away from the driving interface; (iii) first and second arms, extending from the face, each of the first and second arms including: (A) a root portion extending away from the face; and/or (B) a transverse portion extending from the root portion, and across the face, each terminating at an arm end, the root portion of the first and second arms being on opposing sides of the face, such that the arm ends of the transverse portions of the first and second arms are directed away from each other and the transverse portions are substantially parallel to one another; and/or (iv) a gap formed between the transverse portions of the first and second arms, substantially parallel to the transverse portions of the first and second arms.

Example 41. The kit according to example 40, wherein the helical tissue-engaging element and the head are sterile.

Example 42. The kit according to any one of examples 40 to 41, wherein the chord is a diameter of the circular projection of the helical portion.

Example 43. The kit according to any one of examples 40 to 42, wherein a longitudinal axis of the gap is perpendicular to a longitudinal axis of the head.

Example 44. The kit according to any one of examples 40 to 43, wherein the head includes a circumferential groove.

Example 45. The kit according to example 44, further comprising: (i) a cap including a plurality of holes disposed along a circumference thereof; and/or (ii) a pin, wherein the cap is adapted to be disposed about the head, with the pin extending through one of the plurality of holes, within the circumferential groove, and through another one of the plurality of holes.

Example 46. The kit according to example 45, wherein the cap defines a hollow and includes a portal adapted to enable access to the hollow.

Example 47. A system for manufacturing a tissue anchor, the tissue anchor including (i) a helical tissue-engaging element including a helical portion defining a central lumen, and a transverse bar extending from an end of the helical portion, and (ii) an anchor head having first and second arms extending from a face thereof, each of the first and second arms including a root portion extending away from the face and a transverse portion extending from the root portion across the face and terminating at an arm end, the system including: (A) a jig, adapted to receive the anchor head; and/or (B) an elongate pressure applicator, sized and configured to extend through the central lumen of the helical portion, and adapted to apply pressure to the first and second arms, while the anchor head is held within the jig and the transverse bar extends between the face and the transverse portions of the first and second arms, to crimp the transverse bar between the transverse portions of the first and second arms and the anchor head thereby to secure the helical tissue-engaging element to the anchor head.

Example 48. The system according to example 47, wherein the elongate pressure applicator is adapted to apply the pressure along a vector that is colinear with a longitudinal axis of the helical portion, in a manner that crimps the transverse bar between the first and second arms and the face of the anchor head by pressing the transverse portions of the first and second arms longitudinally toward the face of the anchor head and against the transverse bar.

Example 49. The system according to example 48, wherein the vector is colinear with a longitudinal axis of the pressure applicator.

Example 50. A system for manufacturing a tissue anchor, the system comprising: (a) a helical tissue-engaging element, including: (i) a helical portion defining a central lumen; and/or (ii) a transverse bar extending from an end of the helical portion, across a chord of a circular projection of the helical portion; (b) a head, adapted to be attached to the tissue-engaging element, the head comprising: (i) a driving interface; (ii) a face, facing away from the driving interface; (iii) first and second arms, extending from the face, each of the first and second arms including: (A) a root portion extending away from the face; and/or (B) a transverse portion extending from the root portion, across the face and terminating at an arm end, the root portions of the first and second arms being on opposing sides of the face, such that the arm ends of the transverse portions of the first and second arms are directed away from each other and the transverse portions are substantially parallel to one another; and/or (iv) a gap formed between the transverse portions of the first and second arms, substantially parallel to the transverse portions; (c) a jig, adapted to receive the head such that the face and the first and second arms of the head are exposed during manufacture of the tissue anchor; and/or (d) an elongate pressure applicator, sized and configured to extend through the central lumen of the helical portion, and adapted to apply pressure to the first and second arms, while the head is held within the jig and the transverse bar extends between the face and the transverse portions of the first and second arms, to deform the transverse portions of the first and second arms about the transverse bar.

Example 51. The system according to example 50, wherein the elongate pressure applicator is adapted to apply the pressure along a vector that is colinear with a longitudinal axis of the helical portion, in a manner that crimps the transverse bar between the first and second arms and the face of the head by pressing the transverse portions of the first and second arms longitudinally toward the face of the head and against the transverse bar.

Example 52. The system according to example 51, wherein the vector is colinear with a longitudinal axis of the pressure applicator.

Example 53. The system according to any one of examples 50 to 52, wherein the head includes a circumferential groove, the system further comprising: (A) a cap including a plurality of holes disposed along a circumference thereof; and/or (B) a pin, wherein the cap is adapted to be disposed about the head, with the pin extending through one of the plurality of holes, within the circumferential groove, and through another one of the plurality of holes, such that the cap is attached to the head.

Example 54. The system according to example 53, wherein the cap defines a hollow and includes a portal adapted to enable access to the hollow.

Example 55. A method of manufacture of a tissue anchor, the method comprising: (a) obtaining a helical tissue-engaging element, including: (i) a helical portion defining a central lumen; and/or (ii) a transverse bar extending from an end of the helical portion, across a chord of a circular projection of the helical portion; (b) obtaining a head, comprising: (i) a driving interface; (ii) a face facing away from the driving interface; (iii) first and second arms, extending from the face, each of the first and second arms including: (A) a root portion extending away from the face; and/or (B) a transverse portion extending from the root portion, across the face, and terminating at an arm end, the root portions of the first and second arms being on opposing sides of the face, such that the arm ends of the transverse portions of the first and second arms are directed away from each other and the transverse portions are substantially parallel to one another; and/or (iv) a gap formed between the transverse portions of the first and second arms, substantially parallel thereto; (c) inserting the transverse bar into the gap; and/or (d) rotating the helical tissue-engaging element relative to the head, such that a longitudinal axis of the transverse bar is perpendicular to a longitudinal axis of the gap and the transverse bar extends between the face and the transverse portions of the first and second arms.

Example 56. The method according to example 55, further comprising: (e) applying pressure to the first and second arms to deform the transverse portions of the first and second arms about the transverse bar, thereby to secure the head to the tissue-engaging element.

Example 57. The method according to example 56, further comprising, prior to applying the pressure, retaining the head within a jig, such that the first and second arms are accessible, wherein the applying pressure comprises inserting an elongate pressure applicator through the central lumen of the helical portion, to apply pressure to the first and second arms, while the head is retained within the jig and the transverse bar extends between the face and the transverse portions of the first and second arms, to deform the transverse portions of the first and second arms about the transverse bar.

Example 58. The method according to any one of examples 56 to 57, wherein applying the pressure comprises applying pressure along a vector that is colinear with a longitudinal axis of the helical portion of the tissue-engaging element.

Example 59. The method according to any one of examples 56 to 58, wherein applying the pressure initially crimps the transverse bar between the face and an inner surface of the first and second arms, and subsequently causes pivoting of the first and second arms to engage sides of the transverse bar.

Example 60. The method according to any one of examples 55 to 59, further comprising sterilizing the tissue anchor.

Example 61. The method according to any one of examples 55 to 60, wherein the method is devoid of welding.

Example 62. The method according to any one of examples 55 to 61, wherein the head includes a circumferential groove, the method further comprising: (e) disposing a cap about the head, the cap including a plurality of holes along a circumference thereof; and/or (f) attaching the cap to the head by placing a pin through one of the plurality of holes to another of the plurality of holes, via the circumferential groove.

Example 63. The method according to example 62, wherein the attaching comprises attaching the cap to the head such that the cap can rotate relative to the head.

Example 64. A tissue anchor, comprising: (A) a tissue-engaging element, shaped as a helix having a plurality of coils that extend helically around a lumen, the helix being cut from a first tubular segment; (B) a head; and/or (C) a neck portion, comprising a second tubular segment, and connecting the head to the tissue-engaging element by: (i) being connected to the head, and/or (ii) extending, from the head, into the lumen.

Example 65. The tissue anchor according to example 64, wherein the tissue anchor is sterile.

Example 66. The tissue anchor according to any one of examples 64 to 65, wherein the neck portion is concentric with the tissue-engaging element, such that a longitudinal hollow is defined along longitudinal axes of the neck portion and the tissue-engaging element.

Example 67. The tissue anchor according to any one of examples 64 to 66, wherein a distal end of the neck portion is frictionally held within a proximal end of the tissue-engaging element.

Example 68. The tissue anchor according to any one of examples 64 to 66, wherein a distal end of the neck portion is soldered or adhered to a proximal end of the tissue-engaging element.

Example 69. The tissue anchor according to any one of examples 64 to 68, wherein a distal end of the tissue-engaging element comprises a sharp tip adapted to penetrate into tissue.

Example 70. The tissue anchor according to any one of examples 64 to 69, wherein a distal end of the neck portion comprises a sharp tip adapted to puncture or penetrate into tissue.

Example 71. The tissue anchor according to any one of examples 64 to 70, wherein at least part of an external surface of the neck portion includes a recessed thread.

Example 72. The tissue anchor according to example 71, wherein some of the plurality of coils of the helix are disposed within the recessed thread.

Example 73. The tissue anchor according to any one of examples 64 to 70, wherein an external diameter of the second tubular segment is not greater than an internal diameter of the first tubular segment.

Example 74. The tissue anchor according to any one of examples 64 to 73, wherein the distal end of the tissue-engaging element is adapted to transfer strain, from a surface being engaged, to the neck portion.

Example 75. The tissue anchor according to any one of examples 64 to 74, wherein at least one of the first tubular segment and the second tubular segment comprises a hypotube.

Example 76. The tissue anchor according to any one of examples 64 to 75, wherein the helix is laser-cut from the first tubular segment.

Example 77. A method of manufacture of a tissue anchor, the method comprising: (a) cutting a first tubular segment to form a helix having a plurality of coils that extend helically around a lumen, thereby to form a tissue-engaging element; (b) connecting distal end of a second tubular segment into the lumen at a proximal end of the tissue-engaging element.

Example 78. The method according to example 77, wherein a proximal end of the second tubular element includes a head of the tissue anchor.

Example 79. The method according to example 77, further comprising attaching a proximal end of the second tubular element to a head.

Example 80. The method according to any one of examples 77 to 79, further comprising sterilizing the tissue anchor.

Example 81. The method according to any one of examples 77 to 80, wherein the cutting comprises laser cutting.

Example 82. The method according to any one of examples 77 to 81, wherein the cutting comprises cutting the first tubular segment along an entire longitudinal length thereof.

Example 83. The method according to any one of examples 77 to 82, wherein the connecting comprises connecting the second tubular segment to the tissue-engaging element such that a longitudinal hollow is defined along longitudinal axes of the second tubular segment and of the tissue-engaging element.

Example 84. The method according to any one of examples 77 to 83, wherein the connecting comprises frictionally connecting the distal end of the second tubular segment within the proximal end of the tissue-engaging element.

Example 85. The method according to any one of examples 77 to 83, wherein the connecting comprises soldering or adhering the distal end of the second tubular segment to the proximal end of the tissue-engaging element.

Example 86. The method according to any one of examples 77 to 83, wherein at least part of an external surface of the second tubular segment includes a recessed thread, and wherein the connecting comprises disposing some of the plurality of coils of the helix within the recessed thread.

Example 87. The method according to example 86, further comprising, prior to the connecting, etching the recessed thread into the external surface of the second tubular segment.

Example 88. The method according to any one of examples 77 to 87, further comprising sharpening the distal end of the tissue-engaging element to form a sharp tip adapted to penetrate into tissue.

Example 89. The method according to any one of examples 77 to 88, further comprising sharpening the distal end of the second tubular segment to form a sharp tip adapted to puncture or penetrate into tissue.

Example 90. Apparatus, usable with a tissue of a subject or simulation, the apparatus comprising: (a) a tissue anchor that comprises: (i) a head including a notch, (ii) a tissue-engaging element, extending distally from the head, and configured to be driven into the tissue by rotation of the head; (b) a tubular casing, disposed about the tissue engaging element, at least a portion of the tubular casing being axially-compressible; and/or (c) a locking rod extending proximally (i) from a fixation point at which the locking rod is fixed to the tubular casing, and (ii) past at least the portion of the tubular casing toward the head.

Example 91. The apparatus according to example 90, wherein the tissue anchor is sterile.

Example 92. The apparatus according to any one of examples 90 to 91, wherein the apparatus is sterile.

Example 93. The apparatus according to any one of examples 90 to 92, wherein the tissue anchor further includes a neck portion connecting the head to the tissue-engaging element.

Example 94. The apparatus according to any one of examples 90 to 93, wherein the tissue-engaging element comprises a helical tissue engaging element.

Example 95. The apparatus according to any one of examples 90 to 94, wherein the tissue anchor comprises: (A) a tissue-engaging element, shaped as a helix having a plurality of coils that extend helically around a lumen, the helix being cut from a first tubular segment; (B) a head; and/or (C) a neck portion, comprising a second tubular segment, and connecting the head to the tissue-engaging element by: (i) being connected to the head, and/or (ii) extending, from the head, into the lumen.

Example 96. The apparatus according to any one of examples 90 to 95, wherein the tissue anchor is rotatable relative to the tubular casing.

Example 97. The apparatus according to any one of examples 90 to 96, wherein the tissue-engaging element is rotatable within the tubular casing.

Example 98. The apparatus according to any one of examples 90 to 97, wherein an internal diameter of the tubular casing is smaller than a diameter of the head.

Example 99. The apparatus according to any one of examples 90 to 98, wherein the at least a portion comprises an axially compressible spring.

Example 100. The apparatus according to any one of examples 90 to 99, wherein the tubular casing is structured to resist becoming twisted during axial compression thereof.

Example 101. The apparatus according to any one of examples 90 to 100, wherein the distal end of the tubular casing is textured, to create friction between the tubular casing and surface of the tissue and to prevent rotation of the tubular casing relative to the tissue during rotational driving of the tissue anchor into the tissue.

Example 102. The apparatus according to any one of examples 90 to 101, wherein the locking rod is sized and configured, when the tissue anchor is driven into a tissue, to extend through the notch in the head, thereby to prevent rotation of the head relative to the tubular casing.

Example 103. The apparatus according to any one of examples 90 to 102, wherein a longitudinal length of the locking rod is not greater than a longitudinal length of the tubular casing, when the tubular casing is not compressed.

Example 104. The apparatus according to any one of examples 90 to 103, wherein the locking rod is disposed between an interior surface of the tubular casing and an exterior surface of the tissue-engaging element.

Example 105. The apparatus according to any one of examples 90 to 103, wherein the locking rod is disposed alongside an exterior surface of the tubular casing.

Example 106. The apparatus according to example 105, wherein a diameter of the head is greater than a cumulative diameter of the tubular casing and the locking rod, such that the notch is adapted to be vertically aligned, in a specific rotational orientation, with the locking rod, for the locking rod to engage the notch.

Example 107. The apparatus according to any one of examples 90 to 106, wherein the fixation point is adjacent a distal end of the tubular casing.

Example 108. The apparatus according to any one of examples 90 to 107, wherein, in a rest state of the apparatus, the tissue-engaging element extends distally out of the distal end of the tubular casing, the tubular casing is uncompressed, and the proximal end of the tubular casing is longitudinally spaced from the head.

Example 109. The apparatus according to any one of examples 90 to 108, wherein, during anchoring of the tissue anchor into the tissue, the tissue-engaging element extends distally out of the tubular casing and into the tissue and the tubular casing is uncompressed, until the distal end of the tubular casing engages a surface of the tissue, and the head of the tissue anchor engages the proximal end of the tubular casing.

Example 110. The apparatus according to example 109, wherein upon further rotation of the tissue anchor relative to the tubular casing, the tubular casing is adapted to axially compress, to facilitate further extension of the tissue-engaging element into the tissue.

Example 111. The apparatus according to any one of examples 90 to 110, wherein, in an anchored and locked state of the apparatus, the tissue-engaging element extends distally out of the tubular casing and into the tissue, the distal end of the tubular casing engages the exterior surface of the tissue, the tubular casing is compressed such that a proximal end of the locking rod extends beyond the proximal end of the tubular casing and is disposed within the notch of the head, thereby preventing further rotation of the tissue anchor causing motion of the tissue anchor in a distal direction relative to the tubular casing.

Example 112. The apparatus according to example 111, wherein the engagement of the locking rod within the notch facilitates rotation causing motion of the tissue anchor proximally relative to the tubular casing.

Example 113. A kit for manufacture of an apparatus usable with a tissue of a subject or simulation, the kit comprising: (A) a tissue anchor that comprises: (i) a head including a notch; and/or (ii) a tissue-engaging element, extending distally from the head, and configured to be driven into the tissue by rotation of the head; (C) a tubular casing, adapted to be disposed about the tissue-engaging element, the tubular casing including at least a portion which is axially compressible; and/or (D) a locking rod, adapted to be fixed to the tubular casing at a fixation point and to extend proximally therefrom, past at least the portion of the tubular casing, toward the head.

Example 114. The kit according to example 113, wherein the tissue anchor is sterile.

Example 115. The kit according to any one of examples 113 to 114, wherein the tissue anchor, the tubular casing, and the locking rod are sterile.

Example 116. The kit according to any one of examples 113 to 115, wherein the tissue anchor further includes a neck portion connecting the head to the tissue-engaging element.

Example 117. The kit according to any one of examples 113 to 116, wherein the tissue-engaging element comprises a helical tissue engaging element.

Example 118. The kit according to any one of examples 113 to 117, wherein the tissue anchor comprises: (A) a tissue-engaging element, shaped as a helix having a plurality of coils that extend helically around a lumen, the helix being cut from a first tubular segment; (B) a head; and/or (C) a neck portion, comprising a second tubular segment, and connecting the head to the tissue-engaging element by: (i) being connected to the head, and/or (ii) extending, from the head, into the lumen.

Example 119. The kit according to any one of examples 113 to 118, wherein the tissue anchor is adapted to be rotatable relative to the tubular casing.

Example 120. The kit according to any one of examples 113 to 119, wherein the tissue anchor is adapted to be rotatable within the tubular casing.

Example 121. The kit according to any one of examples 113 to 120, wherein an internal diameter of the tubular casing is smaller than a diameter of the head.

Example 122. The kit according to any one of examples 113 to 121, wherein the at least a portion forms an axially compressible spring.

Example 123. The kit according to any one of examples 113 to 122, wherein the tubular casing is structured to resist becoming twisted during axial compression thereof.

Example 124. The kit according to any one of examples 113 to 122, wherein the distal end of the tubular casing is textured.

Example 125. The kit according to any one of examples 113 to 124, wherein the locking rod is sized and configured, when the tissue anchor is driven into a tissue, to extend through the notch in the head, thereby to prevent rotation of the head relative to the tubular casing.

Example 126. The kit according to any one of examples 113 to 125, wherein a longitudinal length of the locking rod is not greater than a longitudinal length of the tubular casing, when the tubular casing is not compressed.

Example 127. The kit according to any one of examples 113 to 126, wherein the locking rod is adapted to be disposed between an interior surface of the tubular casing and an exterior surface of the tissue-engaging element.

Example 128. The kit according to any one of examples 113 to 126, wherein the locking rod is adapted to be disposed alongside an exterior surface of the tubular casing.

Example 129. The kit according to example 128, wherein a diameter of the head is greater than a cumulative diameter of the tubular casing and the locking rod, such that the notch is adapted to be vertically aligned, in a specific rotational orientation, with the locking rod, for the locking rod to engage the notch.

Example 130. A method of manufacture of an apparatus usable with a tissue of a subject or simulation, the method comprising: (a) obtaining a tissue anchor that comprises: (i) a head including a notch; and/or (ii) a tissue-engaging element, extending distally from the head, and configured to be driven into the tissue by rotation of the head; (b) disposing a tubular casing about the tissue-engaging element, at least a portion of the tubular casing being axially-compressible; (c) fixedly anchoring a locking rod to the tubular casing at a fixation point, such that the locking rod extends past at least the portion of the tubular casing toward the head.

Example 131. The method according to example 130, further comprising sterilizing the apparatus.

Example 132. The method according to any one of examples 130 to 131, wherein the obtaining of the tissue anchor comprises a tissue-anchor that further includes a neck portion connecting the head to the tissue-engaging element.

Example 133. The method according to any one of examples 130 to 132, wherein obtaining of the tissue anchor comprises obtaining of a tissue anchor having a helical tissue engaging element.

Example 134. The method according to any one of examples 130 to 133, wherein the obtaining of the tissue anchor comprises obtaining a tissue anchor comprising: (A) a tissue-engaging element, shaped as a helix having a plurality of coils that extend helically around a lumen, the helix being cut from a first tubular segment; (B) a head; and/or (C) a neck portion, comprising a second tubular segment, and connecting the head to the tissue-engaging element by: (i) being connected to the head, and/or (ii) extending, from the head, into the lumen.

Example 135. The method according to any one of examples 130 to 133, wherein the obtaining of the tissue anchor comprises manufacturing the tissue anchor.

Example 136. The method according to example 135, wherein the manufacturing of the tissue anchor comprises: (A) cutting a first tubular segment to form a helix having a plurality of coils that extend helically around a lumen, thereby to form a tissue-engaging element; and/or (B) connecting distal end of a second tubular segment into the lumen at a proximal end of the tissue-engaging element.

Example 137. The method according to any one of examples 130 to 136, wherein the disposing of the tubular casing comprises disposing the tubular casing such that the tissue anchor is rotatable relative to the tubular casing.

Example 138. The method according to any one of examples 130 to 137, wherein the disposing of the tubular casing comprises disposing the tubular casing such that the tissue anchor is rotatable within the tubular casing.

Example 139. The method according to any one of examples 130 to 138, wherein the fixedly anchoring of the locking rod comprises fixedly anchoring the locking rod to an interior surface of the tubular casing, between the tubular casing and the tissue-engaging element.

Example 140. The method according to any one of examples 130 to 138, wherein the fixedly anchoring of the locking rod comprises fixedly anchoring the locking rod to an exterior surface of the tubular casing, such that the locking rod extends longitudinally alongside the tubular casing.

Example 141. The method according to any one of examples 130 to 140, wherein the fixedly anchoring comprises fixedly anchoring the locking rod to the tubular casing at the fixation point, the fixation point being adjacent a distal end of the tubular casing.

Any of the various systems, assemblies, devices, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, apparatus, etc. (e.g., with heat, radiation, ethylene oxide, hydrogen peroxide, etc.).

Any of the treatment techniques, methods, operations, steps, etc. described or suggested herein or in the references incorporated herein can be performed on a living subject (e.g., human, other animal, etc.) or on a non-living simulation, such as a cadaver, cadaver heart, simulator, imaginary person, etc.). When performed on a simulation, the body parts, e.g., heart, tissue, valve, etc., can optionally be referred to as “simulated” (e.g., simulated heart, simulated tissue, simulated valve, etc.) and can comprise, for example, computerized and/or physical representations of body parts, tissue, etc.

The present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

Although the operations of some of the disclosed examples are described in a particular, sequential order for convenient presentation, it should be understood that this manner of description encompasses rearrangement, unless a particular ordering is required by specific language set forth above. For example, operations or steps described sequentially can in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms can vary depending on the particular implementation and are discernible by one of ordinary skill in the art.

What is claimed is:

1. A tissue anchor, comprising:
(a) a helical tissue-engaging element, including:
(i) a helical portion; and
(ii) a transverse bar extending from an end of the helical portion, across a chord of a circular projection of the helical portion; and
(b) a head, attached to the tissue-engaging element, the head comprising:
(i) a driving interface;
(ii) a face, facing away from the driving interface and toward the tissue-engaging element;
(iii) first and second arms, extending from the face, each of the first and second arms including:
(A) a root portion extending away from the face; and
(B) a transverse portion extending from the root portion, and across the face, and terminating at an arm end, the root portions of the first and second arms being on opposing sides of the face, such that the arm ends of the transverse portions of the first and second arms are directed away from each other, and the transverse portions are substantially parallel to one another; and
(iv) a gap formed between the transverse portions of the first and second arms, substantially parallel thereto,
wherein the transverse bar of the tissue-engaging element extends between the face and the first and second arms and is crimped therebetween by deformation of the transverse portions about the transverse bar.

2. The tissue anchor according to claim 1, wherein the first and second arms are oriented in substantially opposite directions to each other.

3. The tissue anchor according to claim 1, wherein the chord is a diameter of the circular projection of the helical portion.

4. The tissue anchor according to claim 1, wherein a longitudinal axis of the gap is perpendicular to a longitudinal axis of the head.

5. The tissue anchor according to claim 1, wherein the tissue-engaging element is secured to the head without welding therebetween.

6. The tissue anchor according to claim 1, wherein the head further includes a circumferential groove.

7. The tissue anchor according to claim 6, further comprising:
a cap including a plurality of holes disposed along a circumference thereof; and
a pin,
wherein the cap is adapted to be disposed about the head, with the pin extending through one of the plurality of holes, within the circumferential groove, and through another one of the plurality of holes, such that the cap is attached to the head.

8. The tissue anchor according to claim 7, wherein the circumferential groove and the pin are dimensioned to allow rotation of the cap relative to the head.

9. The tissue anchor according to claim 7, wherein the cap defines a hollow and includes a portal enabling access to the hollow.

10. A kit for manufacture of a tissue anchor, the kit comprising:
(a) a helical tissue-engaging element, including:
(i) a helical portion; and
(ii) a transverse bar extending from an end of the helical portion, across a chord of a circular projection of the helical portion; and
(b) a head, comprising:
(i) a driving interface;
(ii) a face, facing away from the driving interface;
(iii) first and second arms, extending from the face, each of the first and second arms including:
(A) a root portion extending away from the face; and
(B) a transverse portion extending from the root portion, and across the face, each terminating at an arm end, the root portion of the first and second arms being on opposing sides of the face, such that the arm ends of the transverse portions of the first and second arms are directed away from each other, and the transverse portions are substantially parallel to one another; and
(iv) a gap formed between the transverse portions of the first and second arms, substantially parallel to the transverse portions of the first and second arms.

11. The kit according to claim 10, wherein the chord is a diameter of the circular projection of the helical portion.

12. The kit according to claim 10, wherein a longitudinal axis of the gap is perpendicular to a longitudinal axis of the head.

13. The kit according to claim 10, wherein the head includes a circumferential groove.

14. The kit according to claim 13, further comprising:
a cap including a plurality of holes disposed along a circumference thereof; and
a pin,
wherein the cap is adapted to be disposed about the head, with the pin extending through one of the plurality of holes, within the circumferential groove, and through another one of the plurality of holes.

15. The kit according to claim 14, wherein the cap defines a hollow and includes a portal adapted to enable access to the hollow.

16. The kit according to claim 10, wherein the first and second arms are more malleable than the tissue-engaging element.

17. A system for manufacturing a tissue anchor, the tissue anchor including (i) a helical tissue-engaging element including a helical portion defining a central lumen, and a transverse bar extending from an end of the helical portion, and (ii) an anchor head having first and second arms extending from a face thereof, each of the first and second arms including a root portion extending away from the face and a transverse portion extending from the root portion across the face and terminating at an arm end, the system including:
a jig, adapted to receive the anchor head; and
an elongate pressure applicator, sized and configured to extend through the central lumen of the helical portion, and adapted to apply pressure to the first and second arms, while the anchor head is held within the jig and the transverse bar extends between the face and the transverse portions of the first and second arms, to crimp the transverse bar between the transverse portions of the first and second arms and the anchor head thereby to secure the helical tissue-engaging element to the anchor head.

18. The system according to claim 17, wherein the elongate pressure applicator is adapted to apply the pressure along a vector that is colinear with a longitudinal axis of the helical portion, in a manner that crimps the transverse bar between the first and second arms and the face of the anchor head by pressing the transverse portions of the first and second arms longitudinally toward the face of the anchor head and against the transverse bar.

19. The system according to claim 18, wherein the vector is colinear with a longitudinal axis of the pressure applicator.
